(19) United States
(12) Patent Application Publication
(10) Pub. No.: US 2015/0080292 A1
(43) Pub. Date: Mar. 19, 2015

(54) Cyclic lipopeptide antibiotic Locillomycin (Locillomycin-A, Locillomycin-B, Locillomycin-C) and methods of making and using the same

(71) Applicant: Chuping Luo, (US)

(72) Inventors: Chuping LUO, Nanjing (CN); Zhiyi CHEN, Nanjing (CN); Jun Y. GUO, Ottawa (CA); Xuehui LIU, Nanjing (CN); Xiaoyu WANG, Nanjing (CN); Yongfeng LIU, Nanjing (CN); Youzhou LIU, Nanjing (CN)

(73) Assignee: Chuping Luo, Nanjing (CN)

(21) Appl. No.: 14/190,817

(22) Filed: Feb. 26, 2014

(30) Foreign Application Priority Data
Sep. 13, 2013 (CN) 2013010416487.7

(51) Int. Cl. C07K 7/54; G01N 30/02

(52) U.S. Cl. CPC C07K 7/54; G01N 30/02
USPC 514/2.9; 530/323; 435/71.3; 514/2.3; 514/3.7; 514/3.6; 73/61.52

(57) ABSTRACT

This invention provides new cyclic lipopeptide antibiotic Locillomycin (Locillomycin-A, Locillomycin-B, Locillomycin-C) that display very strong antifungal, antibacterial, antivirus activities in a variety of contexts in vitro; methods of making and using the compounds, wherein Locillomycin-A, Locillomycin-B and Locillomycin-C are derived and purified from the culture of Bacillus subtilis Bs916.

### FIELD OF THE INVENTION

The present invention relates generally to compounds of cyclic lipopeptide antibiotic locillomycin (Locillomycin-A, Locillomycin-B, Locillomycin-C) and methods for making and using to the treatment of infections to fungi, bacteria and virus.

### BACKGROUND OF THE INVENTION

The discovery and application of a cyclic lipopeptide antibiotic started in the 40s of the 20^{th} century, followed by a wide spread of research and application in the areas of medicine, agriculture and animal husbandry and industry due to their very effectiveness to treat infections of fungi, bacteria and virus, and good hemolytic performance. Some of them are of even active anticancer agents. The cyclic lipopeptide antibiotics are mainly derived from microorganisms, especially from Bacillus, and since then, more than 20 different types of them being discovered and used worldwide. These antibiotics have a common structure comprised by a core cyclic peptide with 7 to 10 amino acids and an exocyclic acyl group of hydrophobic fatty acid moiety. According to the chemical structure, they are grouped into 1) Surfacin, which is illustrated as follows:

Surfactin Family

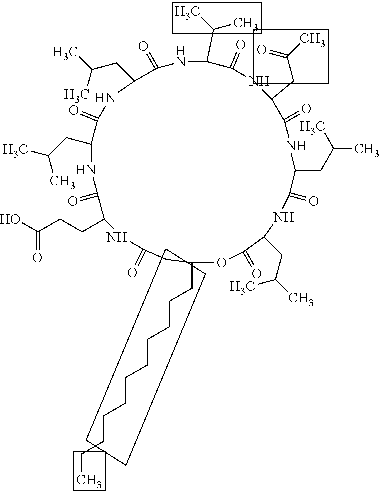 [SMILES: CCCCCCCCCCCC1CC(=O)NC(CCC(=O)O)C(=O)NC(CC(C)C)C(=O)NC(CC(C)C)C(=O)NC(C(C)C)C(=O)NC(CC(C)=O)C(=O)NC(CC(C)C)C(=O)NC(CC(C)C)C(=O)O1]

| Variants | | Length and branching of the acyl chain |
|---|---|---|
| Esperin** | L-Glu-L-Leu-D-Leu-L-Val-L-Asp-D-Leu-L-Leu-COOH | |
| Lichenysin*** | L-XL [symbol] -L-XL [symbol] -D-Leu-L-XL [symbol] -L-Asp-D-Leu-L-XL [symbol] | i-C [symbol] -ai-C [symbol] n-C [symbol] i-C [symbol] ai-C [symbol] |
| Pumilacidin | L-Glu-L-Leu-D-Leu-L-Leu-L-Asp-D-Leu-L-XP [symbol] | |
| Surfactin | L-Glu-L-XS [symbol] -D-Leu-L-XS [symbol] -L-Asp-D-Leu-L-XS [symbol] | i-C [symbol] n-C [symbol] i-C [symbol] ai-C [symbol] |

** the β-carboxyl of Asp5 is engaged in the lactone
*** or halobacillin
XL [symbol] = Gln or Glu;
XL [symbol] = Leu or Ile;
XL [symbol] and XL [symbol] = Val or Ile;
XP [symbol] = Val or Ile;
XS [symbol] = Val, Leu or Ile;
XS [symbol] = Ala, Val, Leu or Ile;
XS [symbol] = Val, Leu or Ile
n, liner 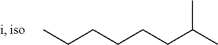 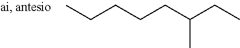 [symbol]
indicates data missing or illegible when filed

2) Iturin, which is illustrated as follows:

Iturin Family

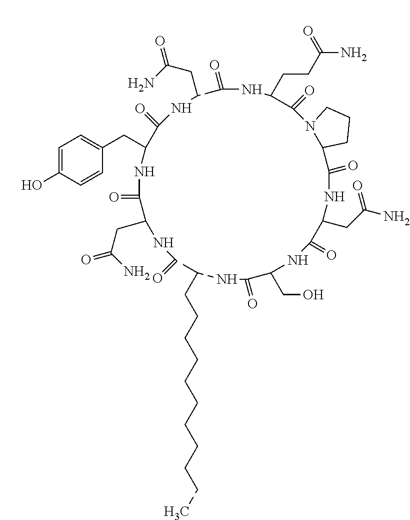 [SMILES: CCCCCCCCCCCC1NC(=O)C(CO)NC(=O)C(CC(N)=O)NC(=O)C2CCCN2C(=O)C(CCC(N)=O)NC(=O)C(CC(N)=O)NC(=O)C(Cc2ccc(O)cc2)NC(=O)C(CC(N)=O)NC1=O]

| | | |
|---|---|---|
| Bacillomycin D | L-Asn-D-Tyr-D-Asn-L-Pro-L-Glu-D-Ser-L-Thr | n-C [symbol] ,i-C [symbol] ,ai-C [symbol] |
| Bacillomycin F | L-Asn-D-Tyr-D-Asn-L-Glu-L-Pro-D-Asn-L-Thr | i-C [symbol] ,i-C [symbol] ,ai-C [symbol] |
| Bacillomycin L | L-Asp-D-Tyr-D-Asn-L-Ser-L-Gln-D-Ser-L-Thr | n-C [symbol] ,i-C [symbol] ,ai-C [symbol] |
| Bacillomycin LC* | L-Asn-D-Tyr-D-Asn-L-Ser-L-Gln-D-Ser-L-Thr | n-C [symbol] ,i-C [symbol] ,ai-C [symbol] ,i-C [symbol] |
| Iturin A | L-Asn-D-Tyr-D-Asn-L-Gln-L-Pro-D-Asn-L-Ser | n-C [symbol] ,i-C [symbol] ,ai-C [symbol] |
| Iturin A | L-Asn-D-Tyr-D-Asn-L-Gln-L-Pro-D-Asn-L-Ser | n-C [symbol] ,i-C [symbol] |
| Iturin C | L-Asp-D-Tyr-D-Asn-L-Gln-L-Glu-D-Asn-L-Ser | n-C [symbol] ,i-C [symbol] ,ai-C [symbol] |
| Mycosubtilin | L-Asn-D-Tyr-D-Asn-L-Gln-L-Pro-D-Ser-L-Asn | n-C [symbol] ,i-C [symbol] ,ai-C [symbol] |

*or bacillopeptin [symbol]
indicates data missing or illegible when filed

3) Fengycin, which is illustrated as follows:

Fengycin Family

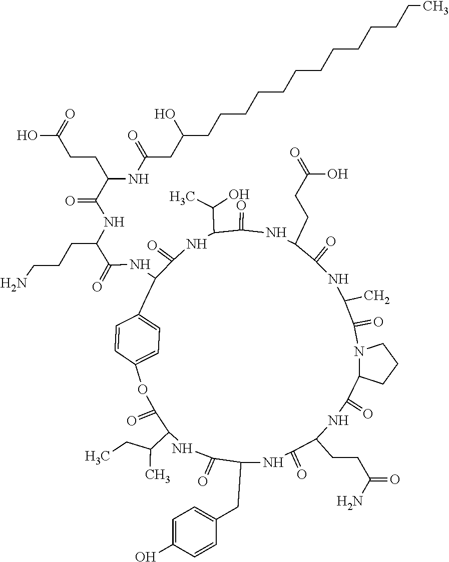 [SMILES: CCCCCCCCCCCCCC(O)CC(=O)NC(CCC(=O)O)C(=O)NC(CCCN)C(=O)NC1Cc2ccc(cc2)OC(=O)C(C(C)CC)NC(=O)C(Cc2ccc(O)cc2)NC(=O)C(CCC(N)=O)NC(=O)C2CCCN2C(=O)C(C)NC(=O)C(CCC(=O)O)NC(=O)C(C(C)O)NC1=O]

| | | |
|---|---|---|
| Fengycin A** | L-Glu-D-Orn-D-Tyr-D- | ai-C₁₅, i-C₁₆, |
| | αThr-L-Glu-D-Ala-L- | n-C₁₆ |
| | Pro-L-Gln-L-Tyr-L-Ile | |
| Fengycin B** | L-Glu-D-Orn-D-Tyr-D- | ai-C₁₅, i-C₁₆, |
| | αThr-L-Glu-D-Val-L- | n-C₁₆,C₁₇ |
| | Pro-L-Gln-L-Tyr-L-Ile | |
| Plipastatin A | L-Glu-D-Orn-L-Tyr-D- | n-C₁₆, ai-C₁₇ |
| | αThr-L-Glu-D-Ala-L- | |
| | Pro-L-Gln-D-Tyr-L-Ile | |
| Plipastatin B | L-Glu-D-Orn-L-Tyr-D- | n-C₁₆, ai-C₁₇ |
| | αThr-L-Glu-D-Val-L- | |
| | Pro-L-Gln-D-Tyr-L-Ile | |

**double bond between carbons 2-3, 3-4, or 13-14 were reported for some acyl chains

The Surfacin family has a polar molecule structure, which is characterized by a core cyclic peptide with 7 amino acid residues and an exocyclic acyl group of β-hydroxyl fatty acid moiety with 13 to 15 carbon atoms. It demonstrates a strong performance of hemolysis and inhibitions of bacterium, virus and cancer cell activities by acting on the phospholipid bilayer of cell membrane. Due to the variability of 7 amino acid residues in the core cyclic peptide, this family provides varieties of antibiotics including Surfacin from Bacillus subtilis, Pumilacidin from Bacillus pumilus and lichenysin from Bacillus licheniformis.

The Iturin family is characterized by a chemical structure comprising of a core cyclic peptide with 7 amino acid residues and an exocyclic acyl group of β-Amino fatty acid moiety with 14 to 17 carbon atoms. It manifests a strong performance of hemolysis and inhibitions of fungus activity by affecting on the surface tension of the cell membrane to form micro pores which cause the leakage of electrolyte and other important ions. However, it shows weak effects on virus and bacteria. This family of antibiotics, including 9 isomers of Iturin-A, Iturin-C, Iturin-D, Iturin-E, Bacillomycin-D, Bacillomycin-F, Bacillomycin-L, Bacillomycin-Lc and Mycosubtilin, is mainly derived from Bacillus subtilis strains and their neighbor species.

The Fengycin family is characterized by a chemical structure comprising of a core cyclic peptide with 10 amino acid residues and an exocyclic acyl group of either saturated or unsaturated β-hydroxyl fatty acid moiety with 14 to 18 carbon atoms. It manifests less effective on the performance of hemolysis than Surfacin family and Iturin family, but more effective on the inhibitions of fungus activity and weaker on the inhibitions of bacteria and virus. This family of antibiotics, including 4 isomers of Fengycin-A, Fengycin-B, Plipastatin-A, Plipastatin-B, is mainly derived from Bacillus subtilis strains and their neighbor species.

This invent provides newly discovered cyclic lipopeptide antibiotic derivatives from the fermentation of Bacillus subtilis Bs916 (CGMCC No. 0808: Classification number in China General Microbiological Culture Collection Center). These derivatives have new structures, which are totally different from known cyclic lipopeptide antibiotics of Surfacin, Iturin and Fengycin, or the report of known antibiotics, and demonstrate strong antibiotic activities against fungi, bacteria and virus. Experiments revealed that the new derivatives had very effectiveness on the inhibition of fungi, bacteria and virus activities, and low side effect, good bioavailability and preparation adaptability, which are all desired characteristics for the potential application.

By the intensive research over the long history since the first discovery of antibiotics, there are tens of thousands of antibiotics in application worldwide. However, pathogens having antibiotic resistance for controlling previously treatable infectious become increasingly common Obviously, there is an urgent need for new antibiotics with novel mechanism of action. The present invention provides all related advantages for such antibiotics.

### DETAILED DESCRIPTION OF THE INVENTION

1. The present invention provides three compounds of cyclic lipopeptide antibiotic locillomycin, defined as Locillomycin-A, Locillomycin-B and Locillomycin-C thereof, according to structural formulae (II):

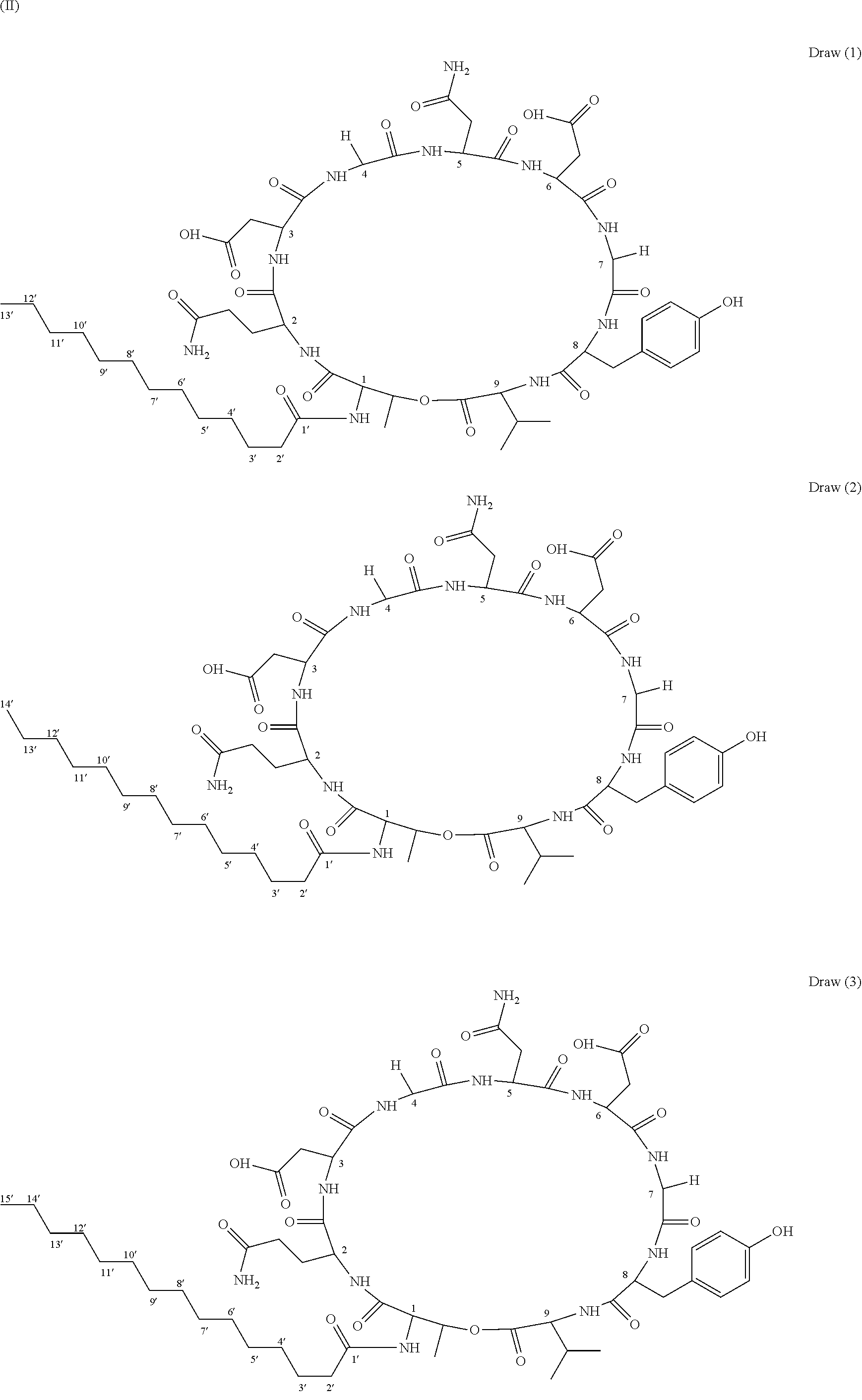 [SMILES: "(II)" ; CCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C ; CCCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C ; CCCCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C]

The definitions are determined according to their chemical structures acquired from the analysis of biochemistry, chromatogram, spectrum and mass spectrum.

The present invent defined the derivatives are in the physical form of a white powder, and in the chemical form of a core cyclic peptide with an attachment of a fatty acid herein, the general chemical composition, after reaction, is CₙHₘNO, wherein the combinations of n and m can be and only be one of the three: n=13 and m=25, or n=14 and m=27, or n=15 and m=29. The “core cyclic peptide” is referred to a chemical structure comprising nine amino acid residues with at least one exocyclic amino acid terminal providing a point of attachment to the straight carbon chain defined above. The nine amino acid residues are connected with a sequence of 1 Thr, 2 D-Gln, 3 L-Asp, 4 L-Gly, 5 L-Asn, 6 L-Asp, 7 L-Gly, 8 L-Tyr, 9 L-Val herein, the β-hydroxy- of the 1 Thr reacted with the carboxyl of the 9 L-Val to form the core cyclic peptide structure via an ester bond. The carboxyl of the fatty acid reacted to the exocyclic amino acid emanating from the 1 Thr to form an amide bond. The derivatives can easily dissolve in methanol and dimethyl sulfoxide, but weakly dissolve in water and ethanol. The differences of the chemical structure among the Locillomycin-A (13 carbon atoms), Locillomycin-B (14 carbon atoms) and Locillomycin-C (15 carbon atoms) only come from the different length of the long-chain acyl group, specifically a methylene (—CH₂) varietion in the side chain of the fatty acid so that Locillomycin-A (C₅₂H₇₉N₁₁O₁₈), Locillomycin-B (C₅₃H₈₁N₁₁O₁₈) and Locillomycin-C(C₅₄H₈₃N₁₁O₁₈).

The present invent provides newly discovered cyclic lipopeptide antibiotic derivatives from the fermentation of Bacillus subtilis Bs916 (CGMCC No. 0808: Classification number in China General Microbiological Culture Collection Center, registered on 30 Sep. 2002).

The present invent follows a route steps below to make and use the compounds thereof,
1, A method for producing Locillomycin-A, Locillomycin-B and Locillomycin-C by fermentation: the fermentation is characterized by culturing Bacillus subtilis Bs916 (CGMCC No. 0808: Classification number in China General Microbiological Culture Collection Center, registered on 30 Sep. 2002) in two stages: firstly, culturing Bs916 in tubes containing LB liquid medium at 37° C. and shaking at 200 r/m on a shaker for 24 hours; secondly, transferring the medium from the first stage into bottles containing LB liquid medium at 28° C. and shaking at 180 r/m on a shaker for 72 hours, then collecting all media to supply to the next route for processing.
2, The separation and purification of the compounds of Locillomycin-A, Locillomycin-B and Locillomycin-C: The processes of separation and purification are characterized by collecting all the fermentation media according to the first step into centrifuge tubes and centrifuging at 5000 r/m for 30 minutes, prior to transferring supernatant into new tubes and adjusting pH to 2.8 and staying at room temperature over night. The supernatant then is centrifuged at room temperature and 8000 r/m for 25 minutes to precipitate, which is followed by two extractions over 48 hours with pure methanol and filtered through a 0.22 μm membrane filter. The filtrate is diluted with deionized water to methanol concentration (v:v) of 30%, and adjusted pH to 7.0, and gravitationally passed through an Amino (NH₂) solid phase extraction (Agilent Technologies, Amino (NH₂)-Box, 6 ml tubes, 500 mg) at room temperature. The subsequent stationary phase is rinsed at room temperature by 10 ml of gradient eluent of a mixture, which is prepared first with deionized water in pure methanol (v/v=50/50), followed by pure methanol, and third by formic acid/methanol v/v=0.5/99.5, then by formic acid/methanol v/v=1/99, and finally by formic acid/methanol v/v=2/98. The eluent of the final rinse with formic acid/methanol v/v=2/98 is collected, and prior to gravitationally passing through a LC-18 solid phase extraction (Agilent Technologies, Supelclean LC-18, 3 ml tubes, 500 mg) at room temperature and adjusting pH to 7.0, drying with pure nitrogen blow, and diluting with deionized water to methanol concentration (v:v) of 30%. The subsequent stationary phase is rinsed at room temperature with 9 ml of gradient eluent of methanol-water solution, which is prepared first with deionized water in pure methanol (v/v=70/30), followed by deionized water/pure methanol (v/v=60/40), third with deionized water/pure methanol v/v=50/50), forth with deionized water/pure methanol v/v=40/60), fifth with deionized water/pure methanol v/v=30/70), finally with deionized water/pure methanol v/v=20/80). The eluents from solutions of deionized water/pure methanol v/v=50/50 and v/v=40/60 are collected, and prior to gravitationally passing through a LC-18 solid phase extraction (Agilent Technologies, Supelclean LC-18, 3 ml tubes, 500 mg) again at room temperature and drying with pure nitrogen blow, and diluting with deionized water to methanol concentration (v:v) of 30%. The subsequent stationary phase is rinsed at room temperature with 30 ml of gradient eluent of methanol-water solution, which is prepared first with deionized water in pure methanol (v/v=64/36), followed by deionized water/pure methanol (v/v=62/38), third with deionized water/pure methanol v/v=60/40), forth with deionized water/pure methanol v/v=58/42), fifth with deionized water/pure methanol v/v=56/44), fifth with deionized water/pure methanol v/v=54/46), fifth with deionized water/pure methanol v/v=52/48), fifth with deionized water/pure methanol v/v=50/50), finally with deionized water/pure methanol v/v=48/52). The eluent from deionized water/pure methanol v/v=60/40 contains compound Locillomycin-A. The eluent from deionized water/pure methanol v/v=56/44 contains compound Locillomycin-B. The eluent from deionized water/pure methanol v/v=52/48 contains compound Locillomycin-C. The eluents are separately dried by pure nitrogen blow and further vacuum dried to obtain final white powder-type compounds.
3, A method for analyzing the compounds of Locillomycin-A, Locillomycin-B and Locillomycin-C: In this step, the process is characterized by using HPLC on a C-18 column (5 μm; 250 by 4.6 mm; VYDAC 218 TP; VYDAC, Hesperia, Calif.) with the acetonitrile-water-trifluoroacetic acid solvent system (50:50:0.5 [vol/vol/vol]) at a flow rate of 0.5 ml min⁻¹. The retention times of 9.0 minutes, 13.0 minutes and 17.8 minutes and UV-visible spectrum at 230 nm are used to identify the Locillomycin-A, Locillomycin-B and Locillomycin-C respectively.
4, A method for appraising the structure of the compounds of Locillomycin-A, Locillomycin-B and Locillomycin-C: In this step, the process is characterized by the system analysis of data derived from UV spectra, amino acid identification, Edman degradation protein sequencing, ¹H-NMR, ¹³C-NMR, ¹³C-edited HSQC, HMBC, COSY, TOCSY, NOESY and/or ROESY. This process is detailed in the section of Example 5.
5, Determination of Antibiotic function for Locillomycin-A, Locillomycin-B and Locillomycin-C: In this step, the process is characterized by antimicrobial experiments, which are conducted by mixing Locillomycin-A, Locillomycin-B and Locillomycin-C separately into different plates containing LB medium and, then, inoculating pathogenic fungi, bacteria and virus into the plates, followed by incubation at 28° C. to check the effectiveness of antifungal (rhizoctonia solani infectious), antibacterial (rice bacterial leaf spot pathogen infectious) functions. The experiments revealed that Locillomycin-A, Locillomycin-B and Locillomycin-C have strong antibiotic activities for inhibition of fungi and bacteria.
6, Test on compositions of acceptable carrier, excipient, diluent for treating infectious of a fungus, or a bacterium, or a virus, or combinations of fungi, bacteria and virus using Locillomycin-A, Locillomycin-B and Locillomycin-C: The acceptable carrier, excipient, diluent refer to as inertia materials to compose either solid, or semi solid, or liquid which can make into powders, tablets, dispersible powders, capsules, suppositories, cream and gel forms in pharmaceutical application. For solid forms, the acceptable carrier, excipient, diluent can be one of, or combinations of, diluent, flavoring agent, solubilizer, lubricant, suspending agent, binder, bulking agent and/or encapsulating material, in certain embodiments of which, they can be one, or combinations of magnesium carbonate, magnesium stearate, talc, sugar, lactose, pectin, dextrin, starch, gelatin, yellow addicted gum, methyl cellulose, sodium carboxymethyl cellulose, low boiling wax and cocoa butter. For powder forms, the acceptable carrier, excipient, diluent can be mixed with 5%-70% (w/w) of bioactive antibiotics, which is micronized in physical size. The liquid forms of the acceptable carrier, excipient, diluent refer to solutions, suspensions and emulsions such as injectable preparations of water and propylene glycol solution for parenteral administration, of which, pH and isotonic property can be adjusted easily. The liquid remedy can also be in the form of polyethylene glycol solution for oral medication after adjusted with coloring agent, flavoring agent, stabilizer and thickener. Other forms of preparation including dispersing bioactive Locillomycin-A, Locillomycin-B and Locillomycin-C in a viscous material such as natural or synthetic gums, or methyl cellulose, or methyl cellulose sodium acid are also acceptable. The doses are normally in the a range of 1 to 1000 mg active antibiotics per unit of delivery carrier, although the forms and compositions can vary.
7, Using Locillomycin-A, Locillomycin-B and Locillomycin-C in the compositions to treat infectious of fungi, bacteria and virus: In the embodiments of present invention, including treatments to the infectious of fungi, bacteria and virus, the results demonstrated a strong effectiveness of antibiotics and wide application possibilities.

### BRIEF DESCRIPTION OF THE DRAWING

FIG. 1. HPLC (High Performance Liquid Chromatographic) peak patterns of Locillomycin-A, Locillomycin-B, Locillomycin-C homologues

FIG. 2. HPLC peak patterns of Locillomycin-A

FIG. 3. HPLC peak patterns of Locillomycin-B

FIG. 4. HPLC peak patterns of Locillomycin-C

FIG. 5. UV spectrum of Locillomycin-A

FIG. 6. UV spectrum of Locillomycin-B

FIG. 7. UV spectrum of Locillomycin-C

FIG. 8 Amino acid analysis of Locillomycin-A, Locillomycin-B, Locillomycin-C by gas chromatography

FIG. 9. DL-amino acid analysis of Locillomycin-A, Locillomycin-B, Locillomycin-C by HPLC

FIG. 10. Edman degradation sequencing Locillomycin-A, Locillomycin-B, Locillomycin-C

FIG. 11. Mass spectrum of Locillomycin-A

FIG. 12. Mass spectrum of Locillomycin-B

FIG. 13. Mass spectrum of Locillomycin-C

FIG. 14. Tandem mass spectrum of Locillomycin-A

FIG. 15. Tandem mass spectrum of Locillomycin-B

FIG. 16. Tandem mass spectrum of Locillomycin-C

FIG. 17. ¹H-NMR spectrum of Locillomycin-A in 90% H₂O/10% D₂O

FIG. 18. TOCSY spectrum of Locillomycin-A in 90% H₂O/10% D₂O

FIG. 19. ROESY spectrum of Locillomycin-A in 90% H₂O/10% D₂O

FIG. 20. ¹³C-edited HSQC spectrum of Locillomycin-A in 90% H₂O/10% D₂O

FIG. 21. ¹H-NMR spectrum of Locillomycin-B in CD₃OH

FIG. 22. ¹³C-NMR spectrum of Locillomycin-B in CD₃OH

FIG. 23. ¹³C-DEPT spectrum of Locillomycin-B in CD₃OH

FIG. 24. ¹H-¹H COSY spectrum of Locillomycin-B in CD₃OH

FIG. 25. TOCSY spectrum of Locillomycin-B in CD₃OH

FIG. 26. ROESY spectrum of Locillomycin-B in CD₃OH

FIG. 27. ¹³C-edited HSQC spectrum of Locillomycin-B in CD₃OH

FIG. 28. HMBC spectrum of Locillomycin-B in CD₃OH

FIG. 29. ¹H-NMR spectrum of Locillomycin-C in CD₃OH

FIG. 30. ¹³C-NMR spectrum of Locillomycin-C in CD₃OH

FIG. 31. ¹H-¹H COSY spectrum of Locillomycin-C in CD₃OH

FIG. 32. TOCSY spectrum of Locillomycin-C in CD₃OH

FIG. 33. ROESY spectrum of Locillomycin-C in CD₃OH

FIG. 34. ¹³C-edited HSQC spectrum of Locillomycin-C in CD₃OH

FIG. 35. HMBC spectrum of Locillomycin-C in CD₃OH

FIG. 36. Sequential assignment and spin system identification of the cyclic peptide

FIG. 37 Important NOE and HMBC signals for the determination of site of the long chain acyl group

FIG. 38. The antifungal activities of Locillomycin-A against Fusarium oxysporum

FIG. 39. The inhibition activities of Locillomycin-A, Locillomycin-B, Locillomycin-C against Porcine epidemic diarrhea virus (PEDV)

### METHODS OF MAKING AND USING THE SAME

The following embodiments provide demonstrations of, but not the limitations of, the present invention:

### Example 1

### The fermentation of Bacillus subtilis Bs916

All together, 80 liters of medium were collected by culturing Bacillus subtilis Bs916 in tubes containing LB liquid medium (10 g tryptone, 5 g yeast extraction, 5 g NaCl per Liter Liquid) at 37° C. and shaking at 200 r/m on a shaker for 24 hours; followed by transferring the medium into bottles each containing 5 ml of the LB liquid medium at 28° C. and shaking at 180 r/m on a shaker for 72 hours.

### Example 2

### Removal of Impurities from the Culturing Medium to Acquire Semi-Purified Antibiotic Mixture

Impurities are removed by centrifuging the collected medium at 5000 r/m for 30 minutes, prior to transferring supernatant into new tubes and adjusting pH to 2.8 and staying at room temperature over night. The supernatant then is centrifuged at room temperature and 8000 r/m for 25 minutes to precipitate, which is followed by two extractions over 48 hours with pure methanol and filtered through a 0.22 μm membrane filter to acquire 200 ml of semi-purified antibiotic mixture.

### Example 3

### Separation of Locillomycin-A, Locillomycin-B, Locillomycin-C from Semi-Purified Antibiotic Mixture

The antibiotic mixture is diluted with deionized water to methanol (v:v) concentration of 30%, and adjusted pH to 7.0, and gravitationally passed through an Amino (NH₂) solid phase extraction (Agilent Technologies, Amino (NH₂)-Box, 6 ml tubes, 500 mg) at room temperature. The subsequent stationary phase is rinsed at room temperature by 10 ml of gradient eluent of a mixture, which is prepared first with deionized water in pure methanol (v/v=50/50), followed by pure methanol, and third by formic acid/methanol v/v=0.5/99.5, then by formic acid/methanol v/v=1/99, and finally by formic acid/methanol v/v=2/98. The eluent of the final rinse with formic acid/methanol v/v=2/98 is collected, and prior to gravitationally passing through a LC-18 solid phase extraction (Agilent Technologies, Supelclean LC-18, 3 ml tubes, 500 mg) at room temperature and adjusting pH to 7.0, drying with pure nitrogen blow, and diluting with deionized water to methanol (v:v) concentration of 30%. The subsequent stationary phase is rinsed at room temperature with 9 ml of gradient eluent of methanol-water solution, which is prepared first with deionized water in pure methanol (v/v=70/30), followed by deionized water/pure methanol (v/v=60/40), third with deionized water/pure methanol v/v=50/50), forth with deionized water/pure methanol v/v=40/60), fifth with deionized water/pure methanol v/v=30/70), finally with deionized water/pure methanol v/v=20/80). The eluents from solutions of deionized water/pure methanol v/v=50/50 and v/v=40/60 are collected, which contained Locillomycin-A, Locillomycin-B and Locillomycin-C mixture about 80 ml at purity of 94.4%.

The collected mixture was diluting with deionized water to methanol (v:v) concentration of 30%, then dried with pure nitrogen blow followed by to gravitationally pass through a LC-18 solid phase extraction (Agilent Technologies, Supelclean LC-18, 3 ml tubes, 500 mg) again at room temperature. The subsequent stationary phase is rinsed at room temperature with 30 ml of gradient eluent of methanol-water solution, which is prepared first with deionized water in pure methanol (v/v=64/36), followed by deionized water/pure methanol (v/v=62/38), third with deionized water/pure methanol v/v=60/40), forth with deionized water/pure methanol v/v=58/42), fifth with deionized water/pure methanol v/v=56/44), fifth with deionized water/pure methanol v/v=54/46), fifth with deionized water/pure methanol v/v=52/48), fifth with deionized water/pure methanol v/v=50/50), finally with deionized water/pure methanol v/v=48/52). The eluent from deionized water/pure methanol v/v=60/40 contains compound Locillomycin-A about 12 mg at purity of 95.7%. The eluent from deionized water/pure methanol v/v=56/44 contains compound Locillomycin-B about 10 mg at purity of 97.8%. The eluent from deionized water/pure methanol v/v=52/48 contains compound Locillomycin-C about 14 mg at purity of 98.8%. The eluents are separately dried by pure nitrogen blow and further vacuum dried to obtain final white powder-type compounds.

### Example 4

### Analyze the Compounds Using HPLC

The final white powder-type compounds were analyzed using HPLC on a C-18 column (5 μm; 250 by 4.6 mm; VYDAC 218 TP; VYDAC, Hesperia, Calif.) with the acetonitrile-water-trifluoroacetic acid solvent system (50:50:0.5 [vol/vol/vol]) at a flow rate of 0.5 ml min⁻¹. The retention times of 9.0 minutes, 13.0 minutes and 17.8 minutes and UV-visible spectrum at 230 nm were used to identify the Locillomycin-A, Locillomycin-B and Locillomycin-C respectively.

### Example 5

### Appraise the Chemical Structures of the Compounds

The final white powder-type compounds were appraised by system analysis with data acquired from UV spectra, amino acid identification, Edman degradation protein sequencing, ¹H-NMR, ¹³C-NMR, ¹³C-edited HSQC, HMBC, COSY, TOCSY, NOESY and/or ROESY. The main physical and chemical properties of Locillomycin-A, Locillomycin-B and Locillomycin-C are listed in Table 1.

TABLE 1
Main physical and chemical properties of Locillomycin-A, Locillomycin-B and
Locillomycin-C

| | Locillomycin-A | Locillomycin-B | Locillomycin-C |
|---|---|---|---|
| Color and Shape | White Powder | White Powder | White Powder |
| Molecule Formula and | C₅₂H₈₀N₁₁O₁₈ | C₅₃H₈₂N₁₁O₁₈ | C₅₄H₈₄N₁₁O₁₈ |
| Molecule Weight | MW = 1145.6 | MW = 1159.6 | MW = 1173.6 |
| UVλmax nm | 230, 280 | 230, 280 | 230, 280 |
| Retention Time (HPLC) | 9.0 min | 13.0 min | 17.8 min |
| Solubility | Methanol, Dimethyl | Methanol, Dimethyl | Methanol, Dimethyl |
| | Sulfoxide, Water | Sulfoxide, Water | Sulfoxide, Water |
| ESI-MS | 1146.6 [M + H] | 1160.6 [M + H] | 1174.6 [M + H] |

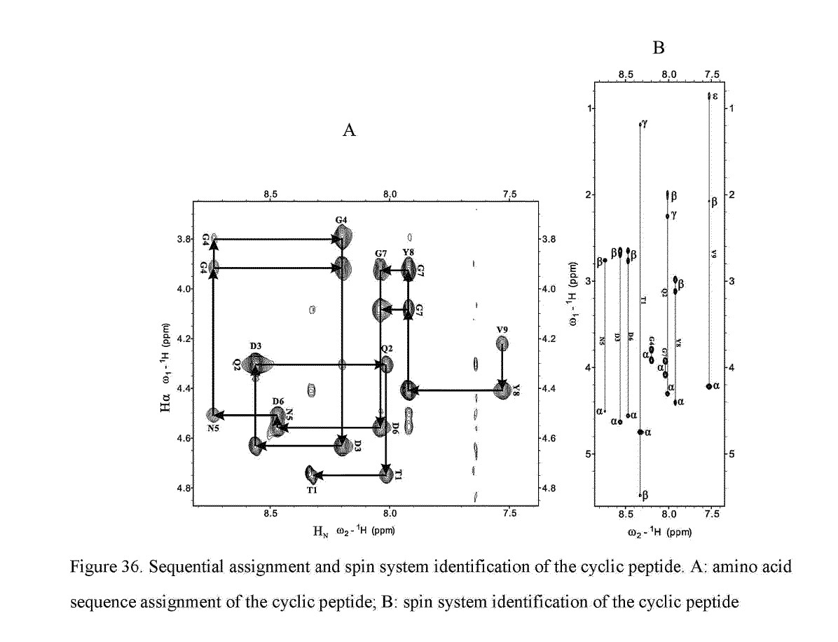

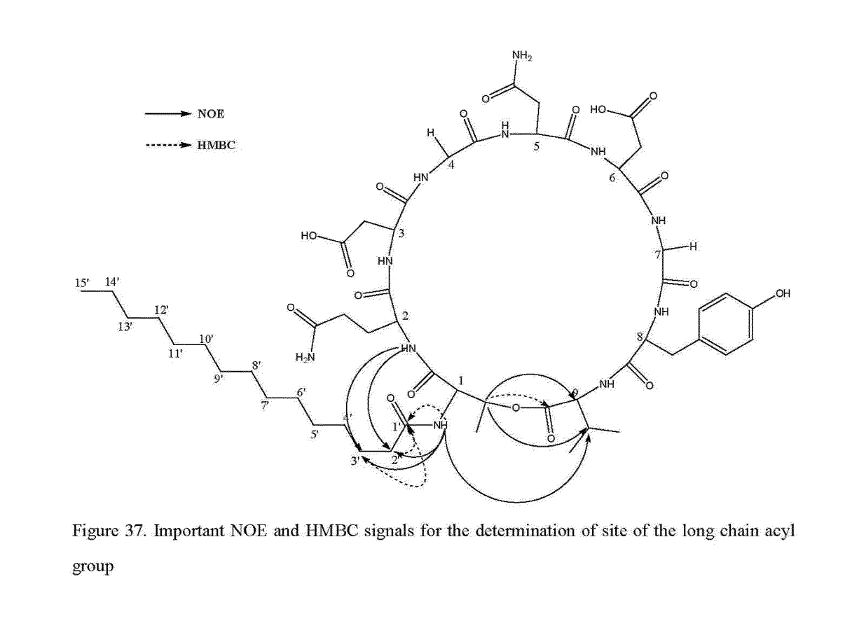

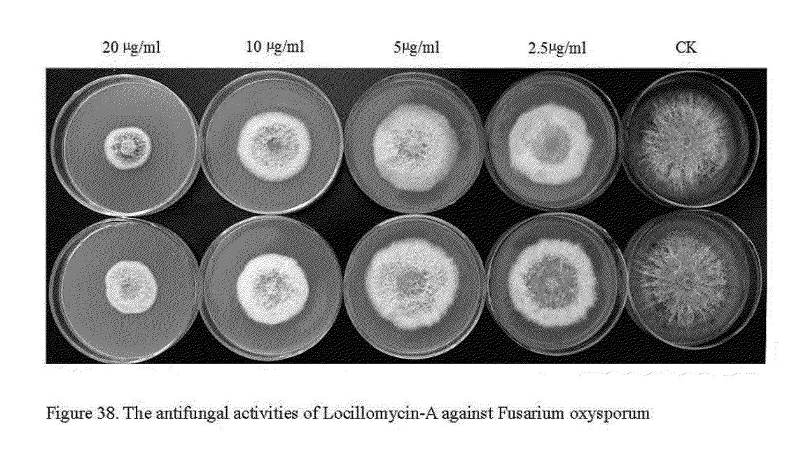

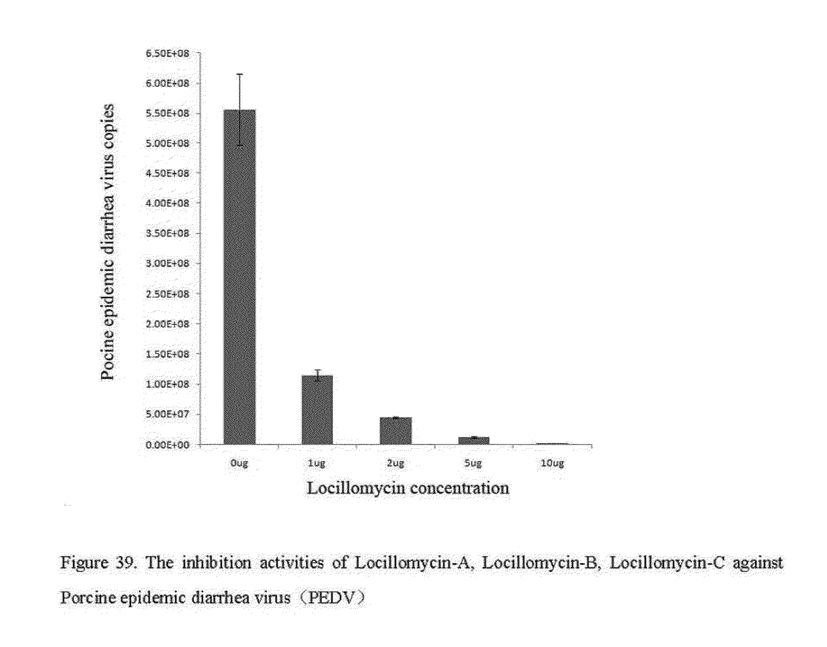

Characterization of all derivatives of Locillomycin (Locillomycin-A, Locillomycin-B, Locillomycin-C) was demonstrated by the signal assigning process and determining the molecular structure of the Locillomycin-C as an example. The assigning process started from connecting the fingerprint area of the ROESY spectrum of Locillomycin-C(H_{α}: 3.6-4.9 ppm, H_{N}: 7.4-8.8 ppm) as shown in the left panel of FIG. 36. Then the TOCSY spectrum was used to identify the different spin systems of the amino acid residues (shown in the right panel of FIG. 36). By using these two spectra, we concluded that there were nine residues in the compound of the Locillomycin-C, which are consistent to the sequencing result of the same derivative. These amino acid residues are connected with a sequence of Thr1-Gln2-Asp3-Gly4-Asn5-Asp6-Gly7-Tyr8-Val9. The characteristic cross peaks of the side chain amide to 3 protons in the ROESY spectrum were used to differentiate the Asn to Asp residues. Several Thr1 to Val9 interactions observed in the ROESY spectrum (solid-line arrows in FIG. 37) show that these two residues are close in space, indicating a cyclic structure connected end-to-end. From the large peaks around 1.2-1.4 ppm in the ¹H spectrum, we conclude that a long alkyl group is attached to the cyclic peptide structure.

By using a ¹³C-edited (CH₂ negative, CH₃, CH positive) HSQC spectrum, protonated carbons were assigned after the assignment of most of the protons. So far, all known pieces of the compound contributed to 13 carbonyl signals while we observed 14 carbonyl signals in the ¹³C spectrum. This indicated that there must be a carbonyl group in the long alkyl chain of this compound. The analysis of the HMBC spectrum resolved that this carbonyl signal was 177.27 ppm. The HMBC spectrum also had a cross peak between this carbonyl carbon and amide proton of the Thr1 (dotted-line arrow in FIG. 37). When cross peak of beta protons of Thr1 (5.48 ppm) to carbonyl group of Val9 (171.32 ppm) was observed in the HMBC, there was no cross peak observed from amide proton of Thr1 to carbonyl of Val9 (FIG. 37). What was more, beta proton of Thr1 also showed interactions to alpha and beta protons of Val9 in the ROESY spectrum (FIG. 36). From the above information, we could be able to figure out the attached site of the alkyl chain together with the carbonyl group (actually a long chain acyl group) and the pattern of the end-to-end connection of Thr1 to Val9 (FIG. 36). From the chemical shift information provided by the ¹H and ¹³C spectra and connectivities provided by the ROESY, TOCSY and HMBC spectra, we could predict the structure of the long chain acyl group as shown in FIG. 36. We concluded the number of CH₂ groups by comparing our resolved structure pieces of the compound and its molecular weight from the Mass Spectrum and confirmed this number by the integration of the ¹H spectrum of the multi-CH₂ area. No branching was identified from NMR or MS. Thus, the molecular structure of the Locillomycin-C was identified by a complex analysis of its biochemical, chromatographical and different types of spectral data.

As noted above, the present invention provides cyclic lipopeptide antibiotic derivatives thereof, and uses thereof. The cyclic lipopeptide antibiotic derivatives of the present invention have a “core cyclic peptide” including at least one exocyclic amino acid indicated by a dashed line, which is illustrated as follows:

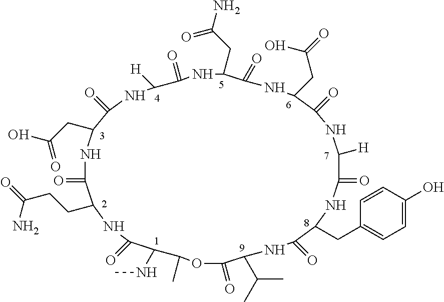

In the above core cyclic peptide moiety, the dashed line emanating from the exocyclic amino acid indicates the point of attachment of a straight carbon chain herein, the general chemical composition is CₙHₘNO, wherein the combinations of n and m can be and only be one of the three: n=13 and m=25, or n=14 and m=27, or n=15 and m=29. The cyclic lipopeptide antibiotic derivatives of the present invention thus are defined as locillomycin lipopeptide antibiotics, and according to their carbon chain properties, further as Locillomycin-A (C₁₃H₂₅NO), Locillomycin-B (C₁₄H₂₇NO) and Locillomycin-C(C₁₅H₂₉NO). In this embodiment, the compounds of locillomycin lipopeptide antibiotic are derived from fermentation of Bacillus subtilis Bs916 (CGMCC No. 0808: Classification number in China General Microbiological Culture Collection Center), and followed by a process of purification.

As used herein, “locillomycin lipopeptide antibiotic” refers to an antibiotic comprising a cyclic peptide core that includes an exocyclic amino acid having a side chain with a primary fatty acid moiety.

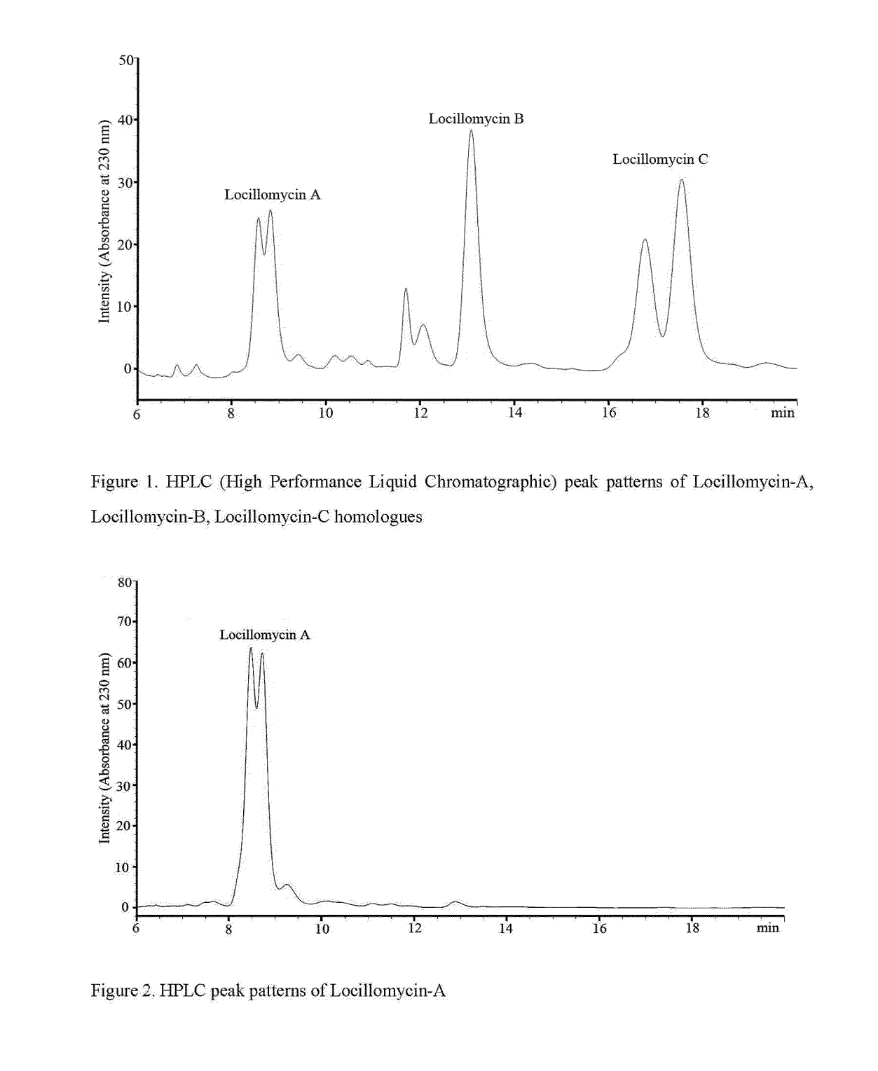

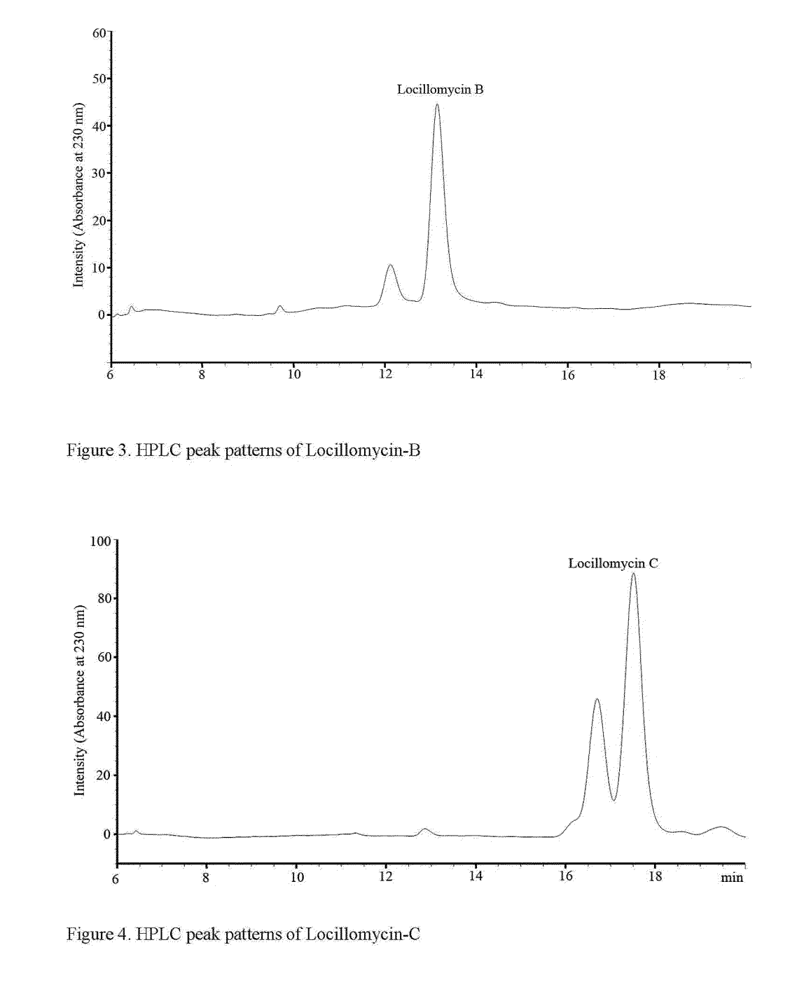

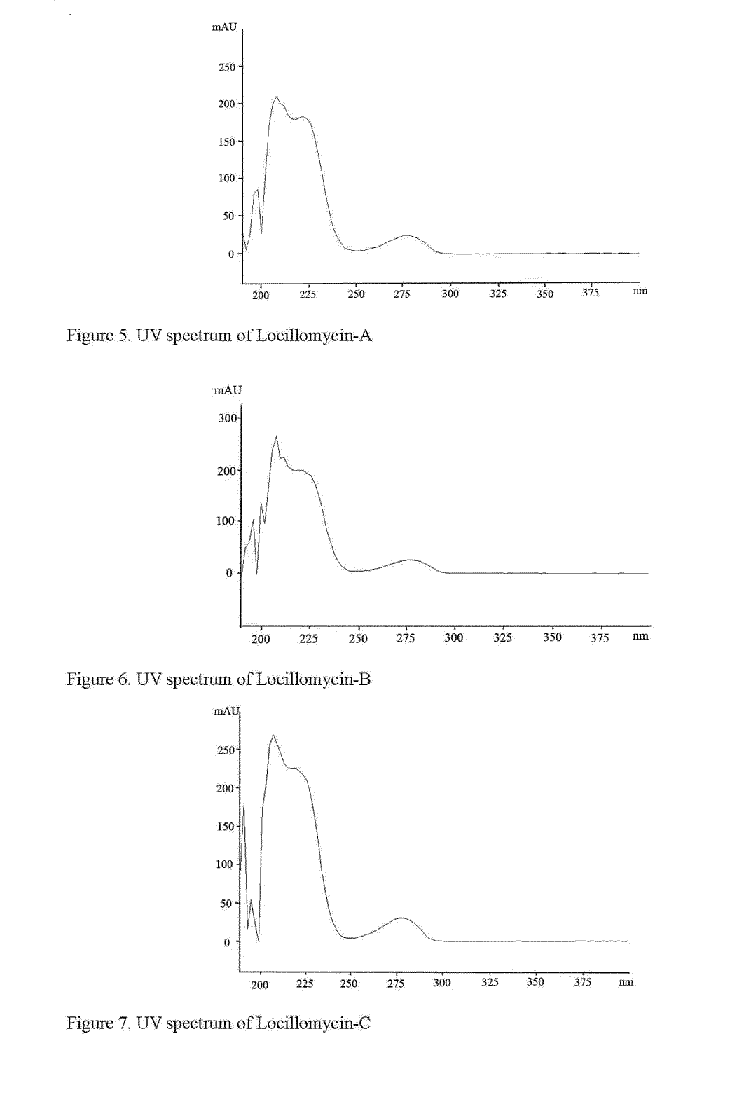

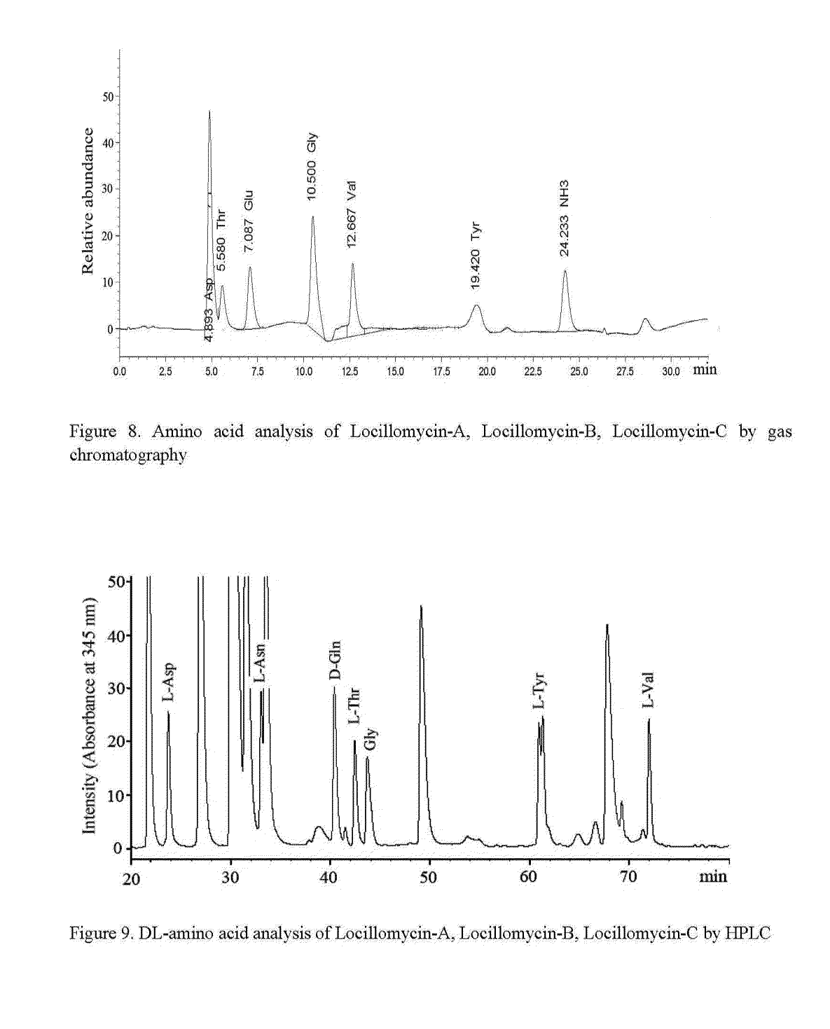

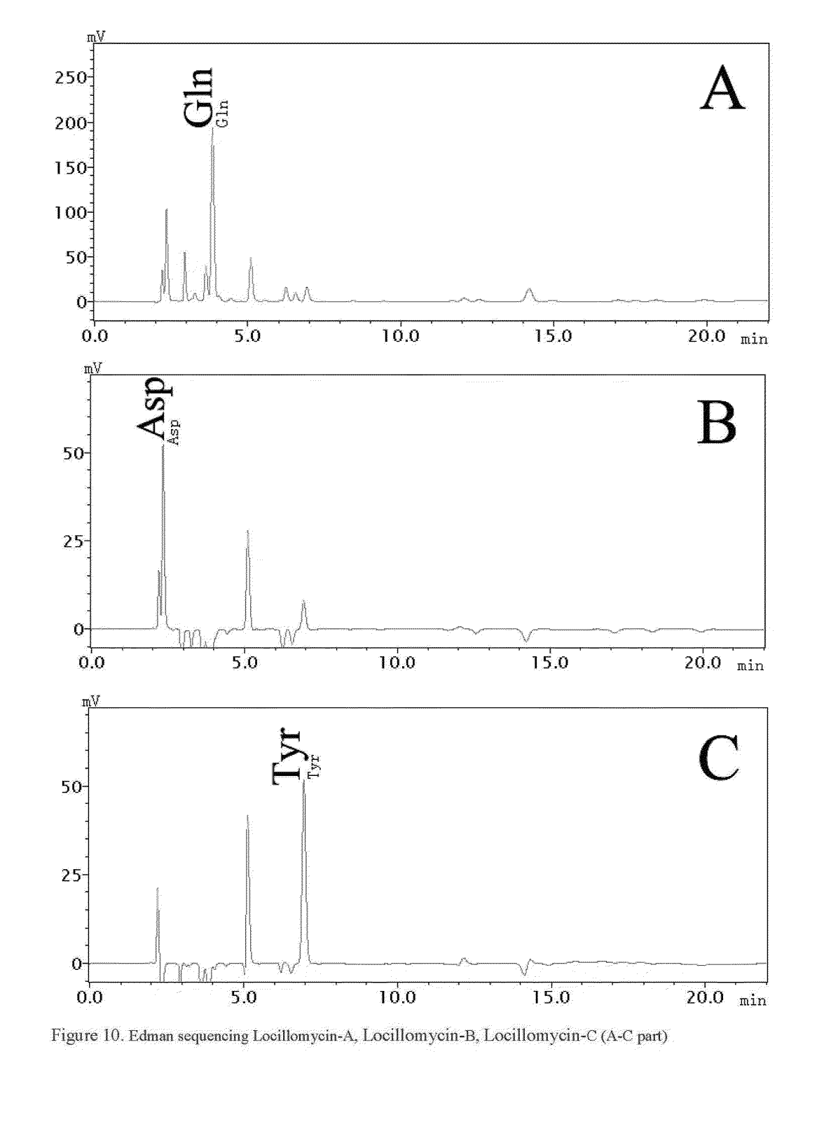

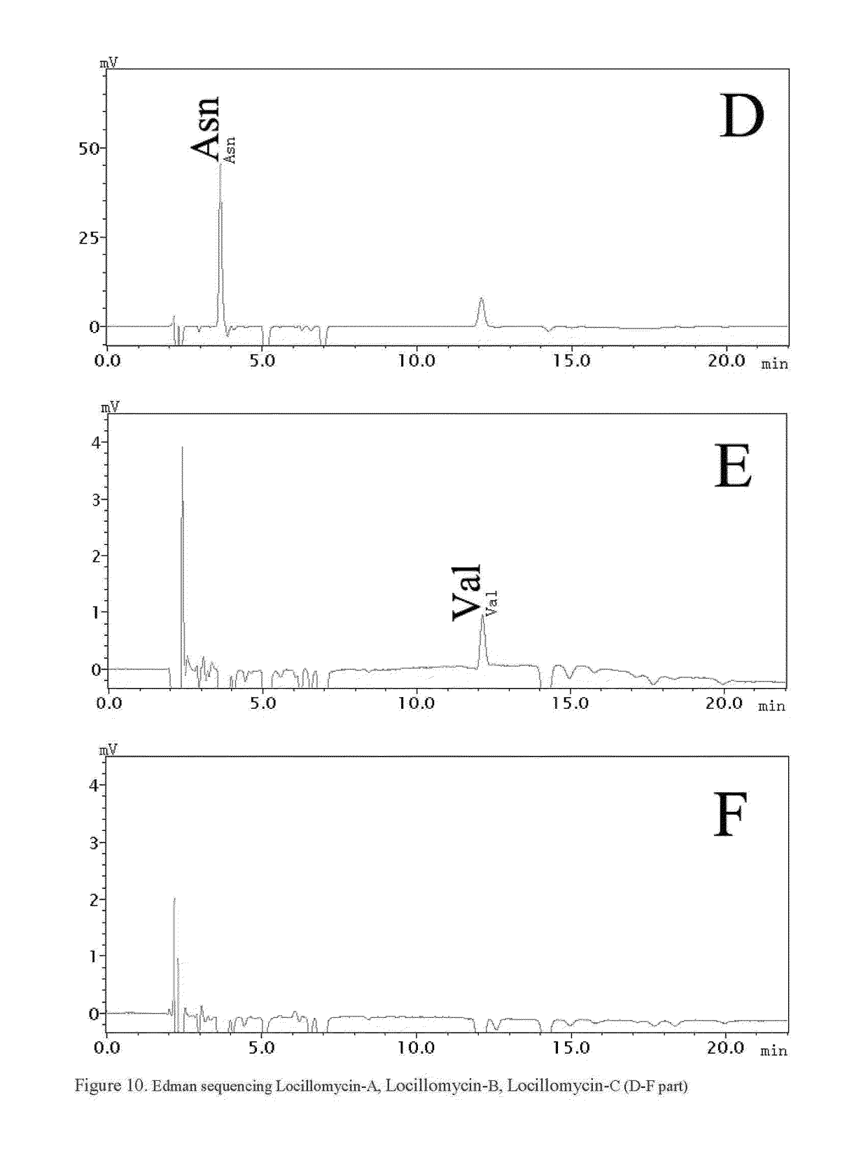

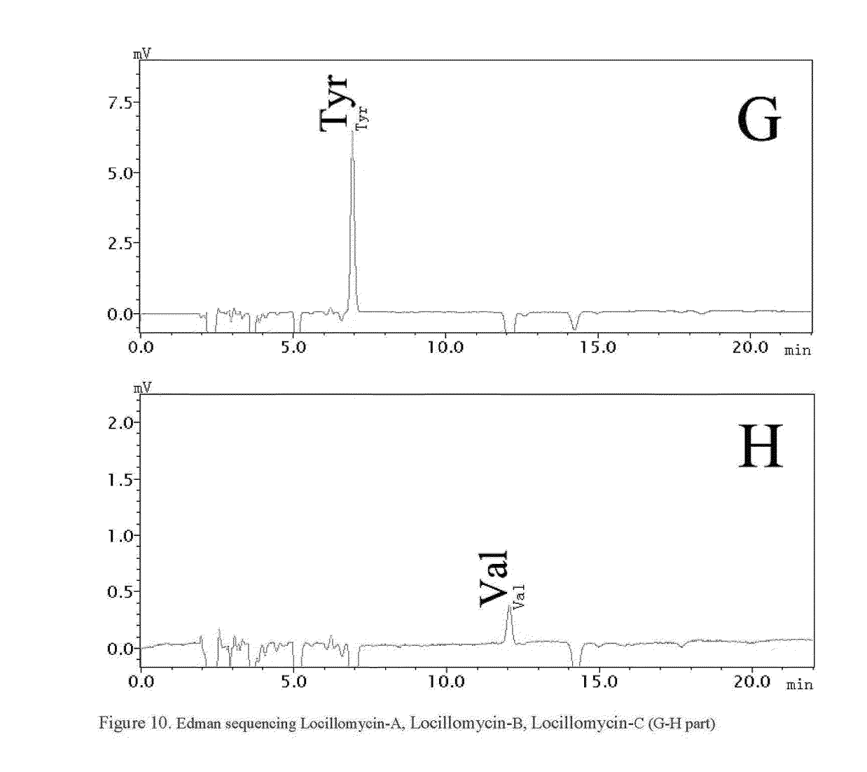

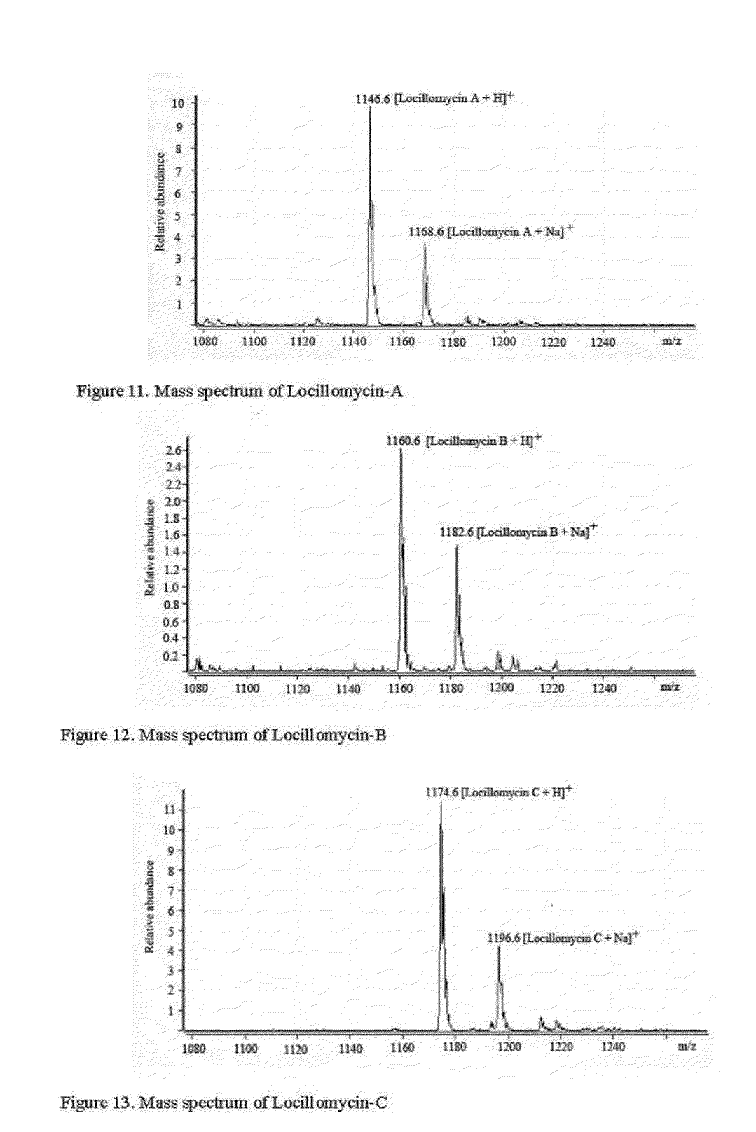

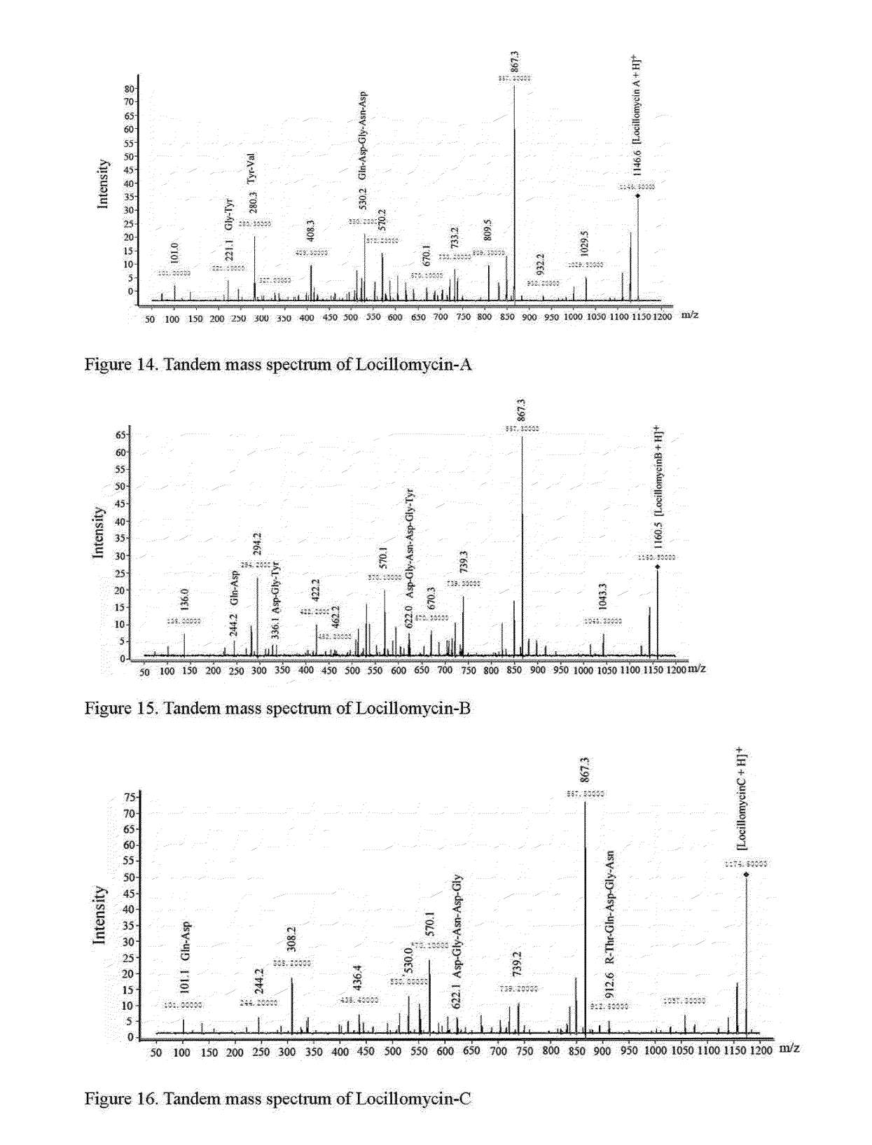

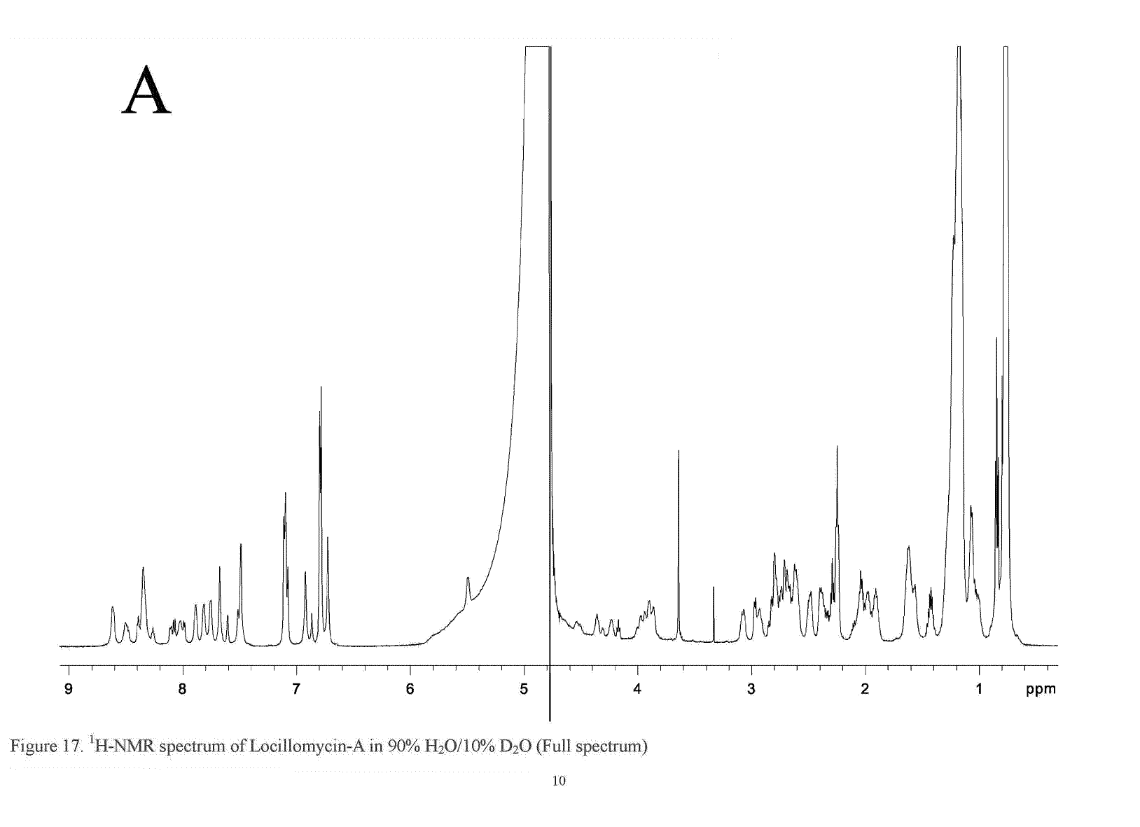

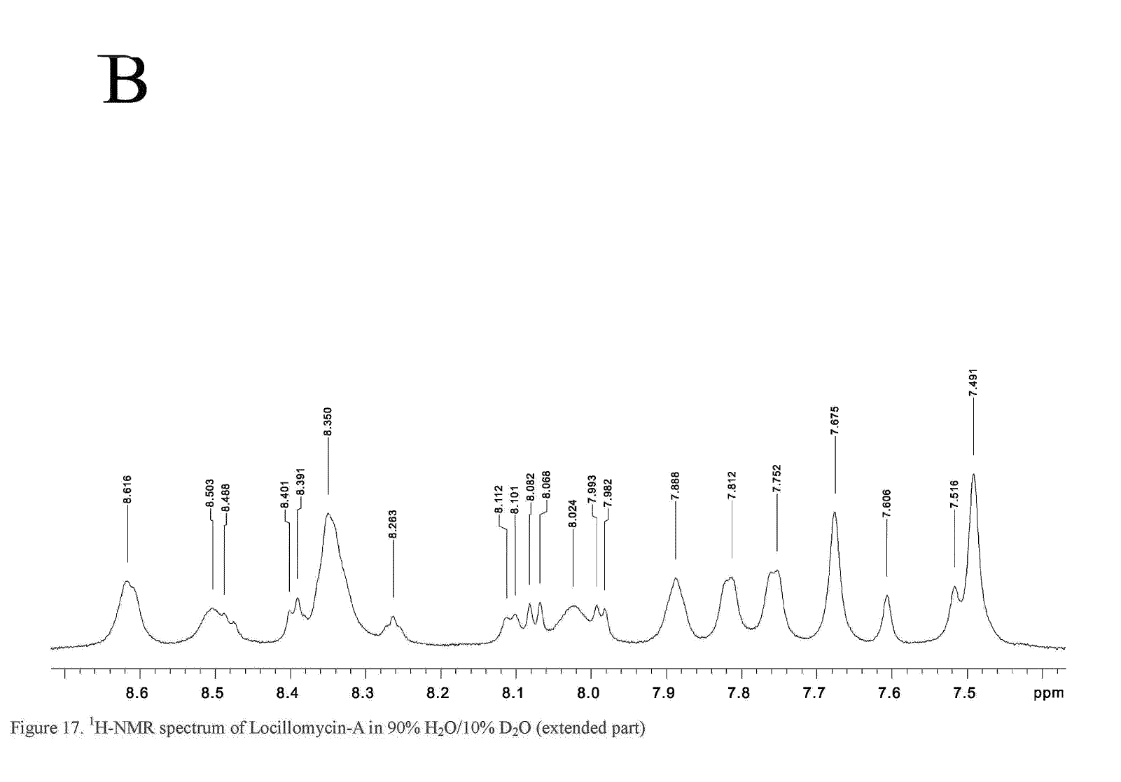

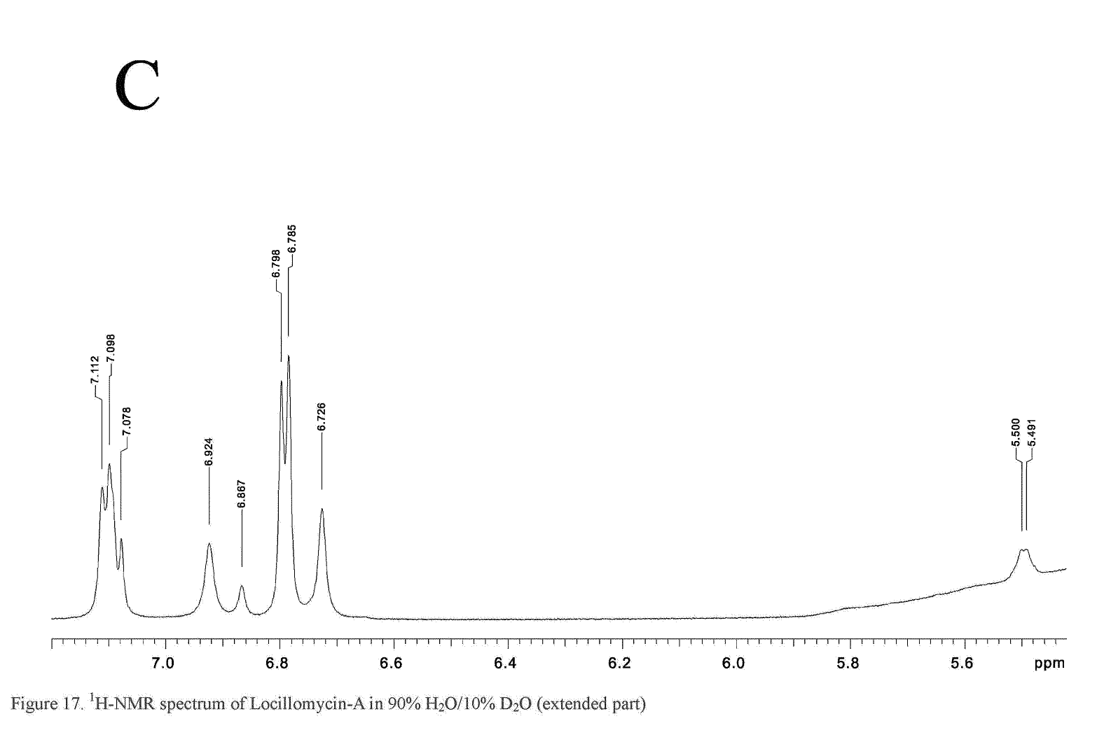

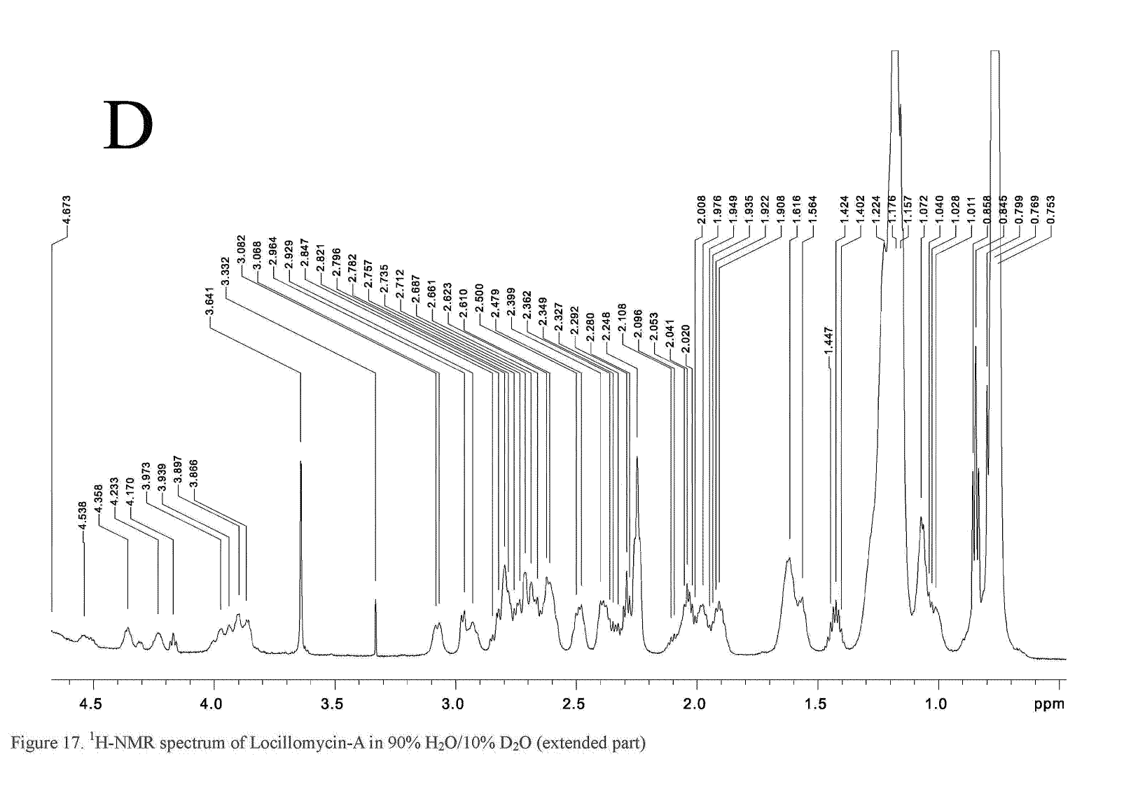

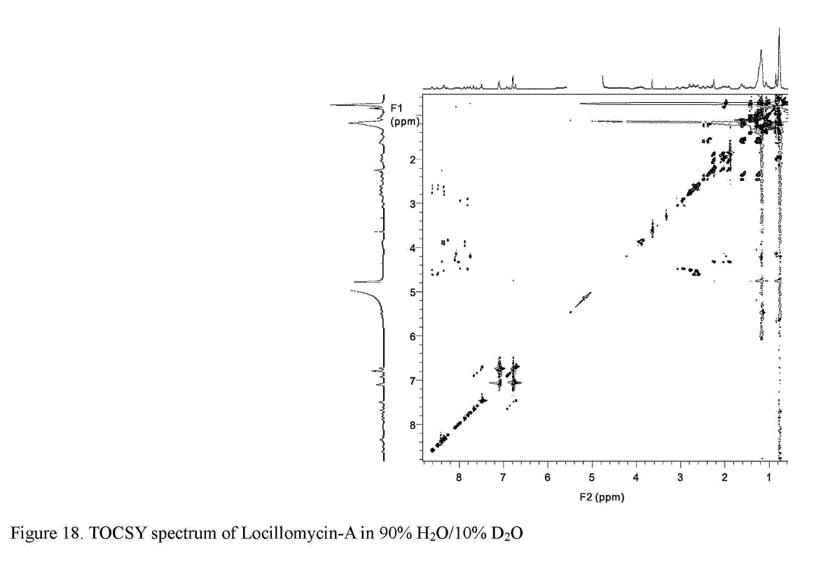

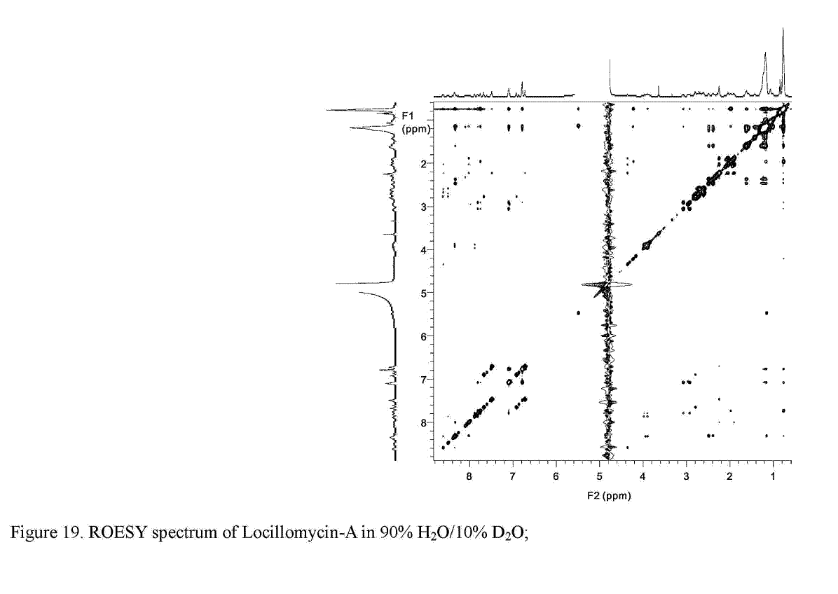

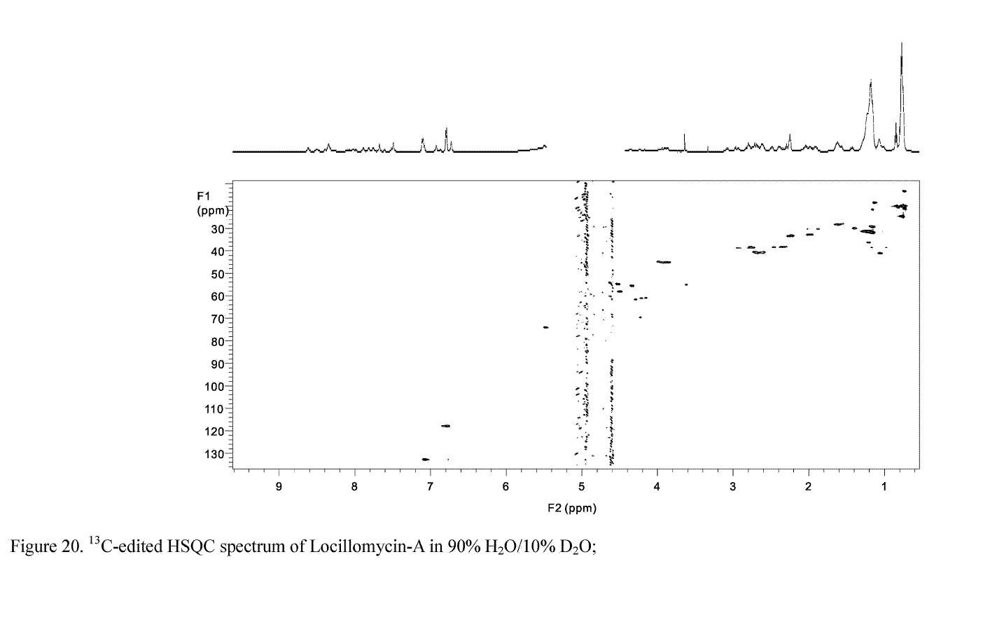

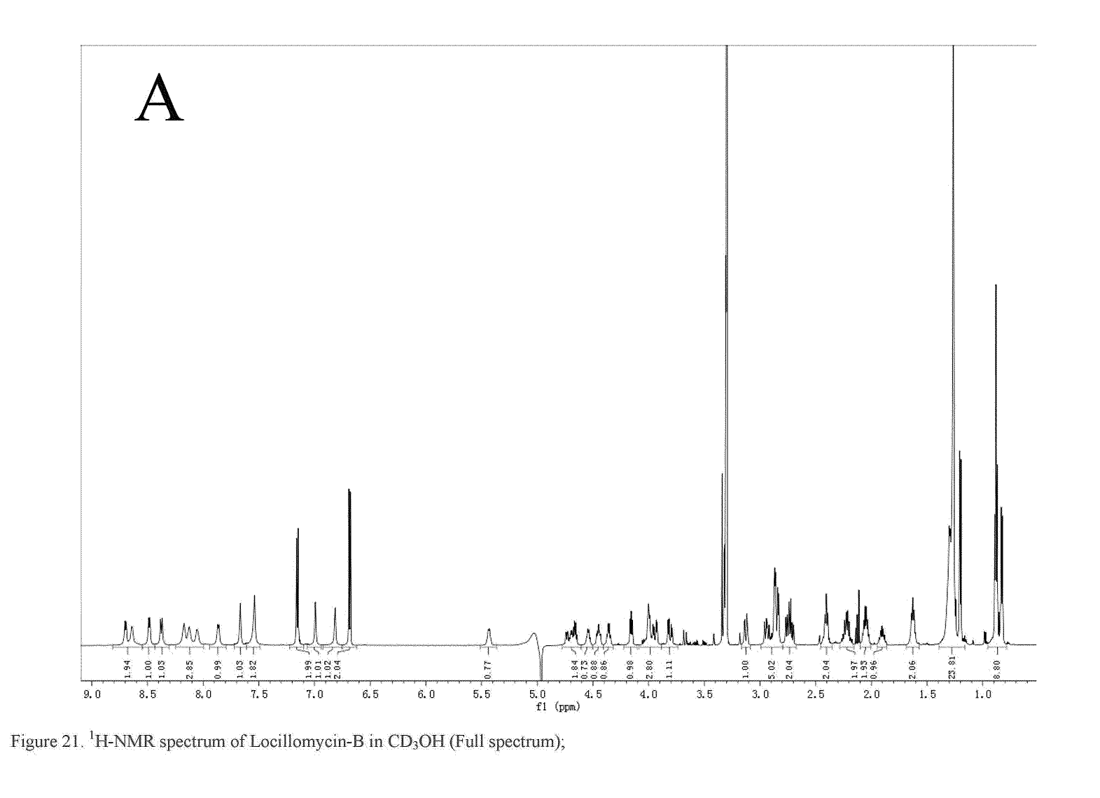

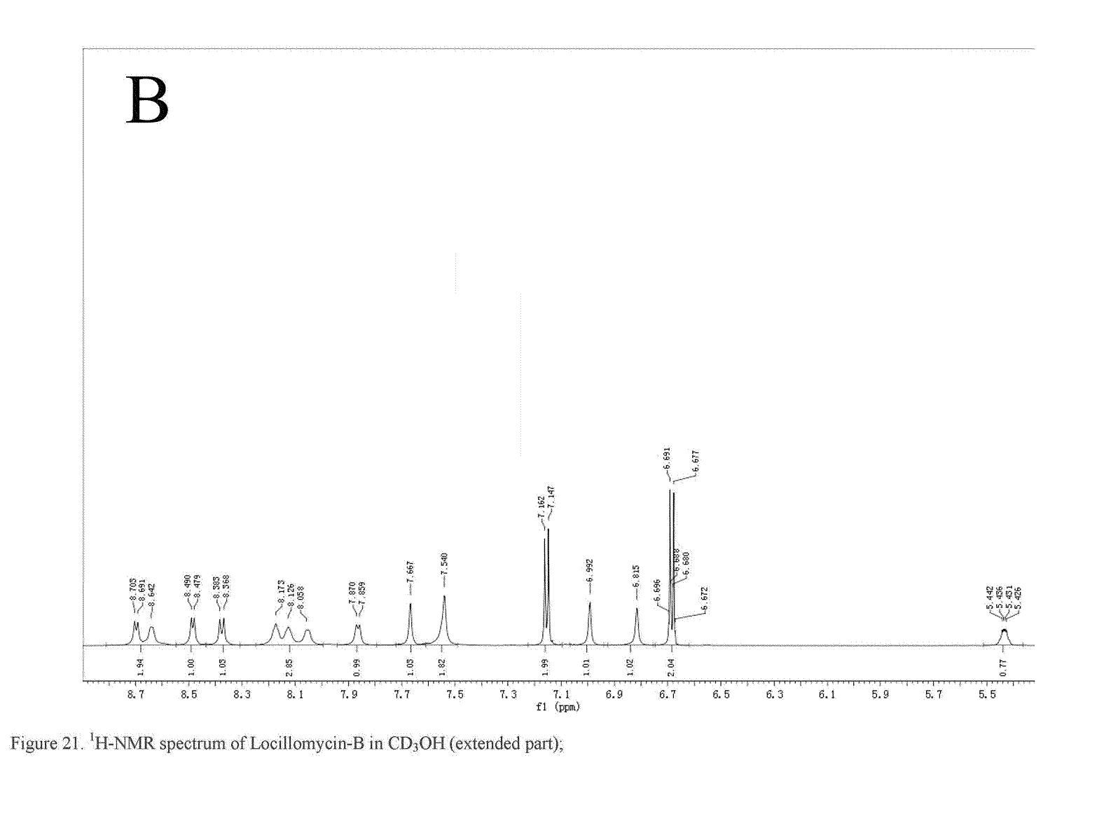

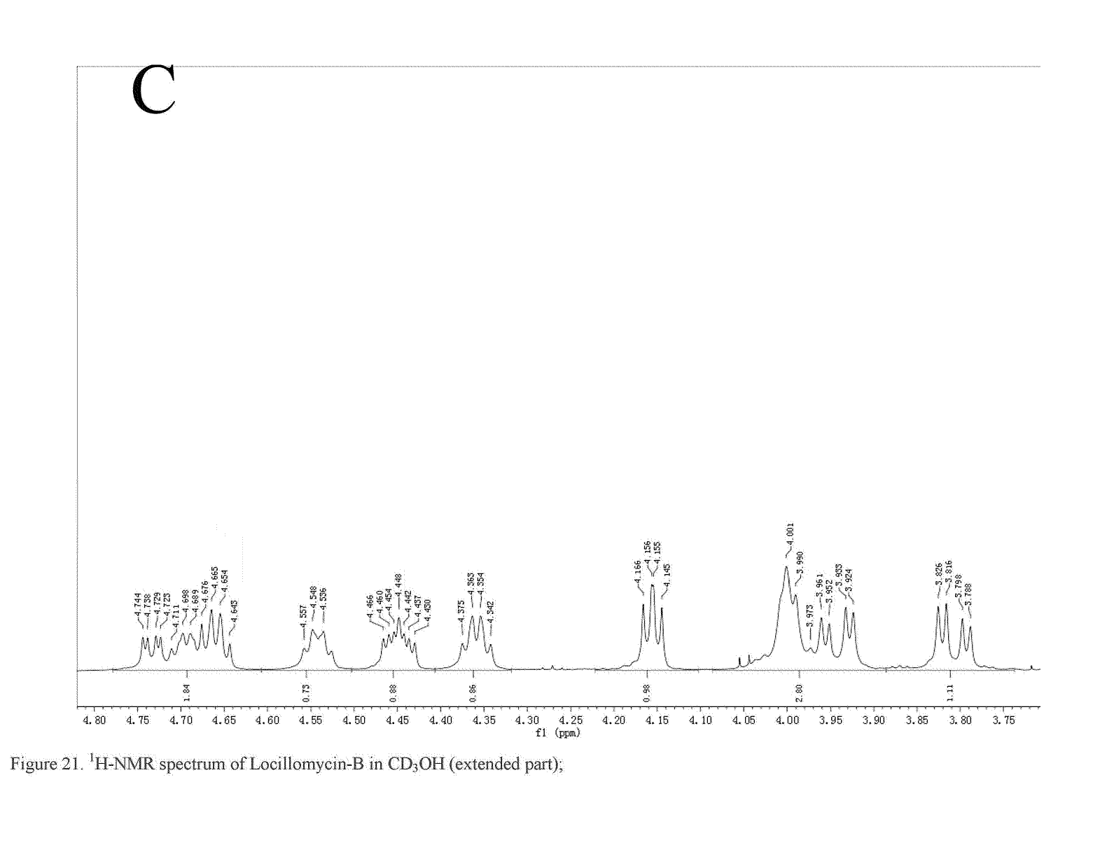

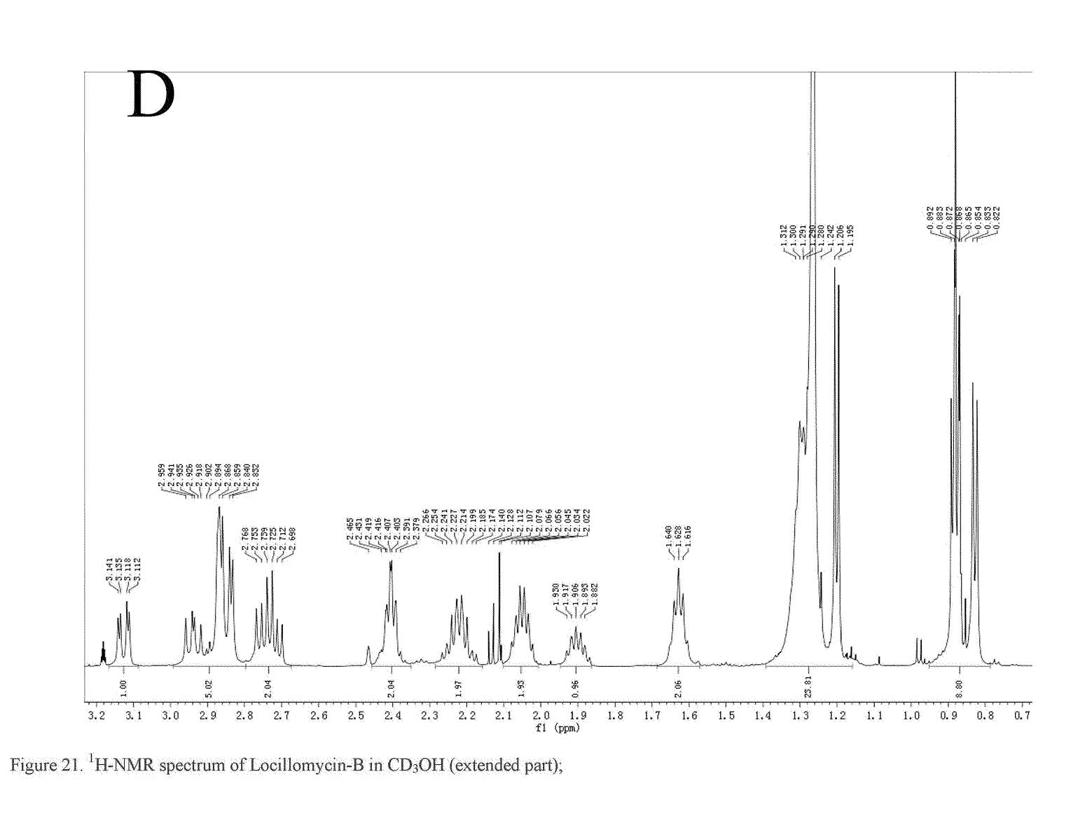

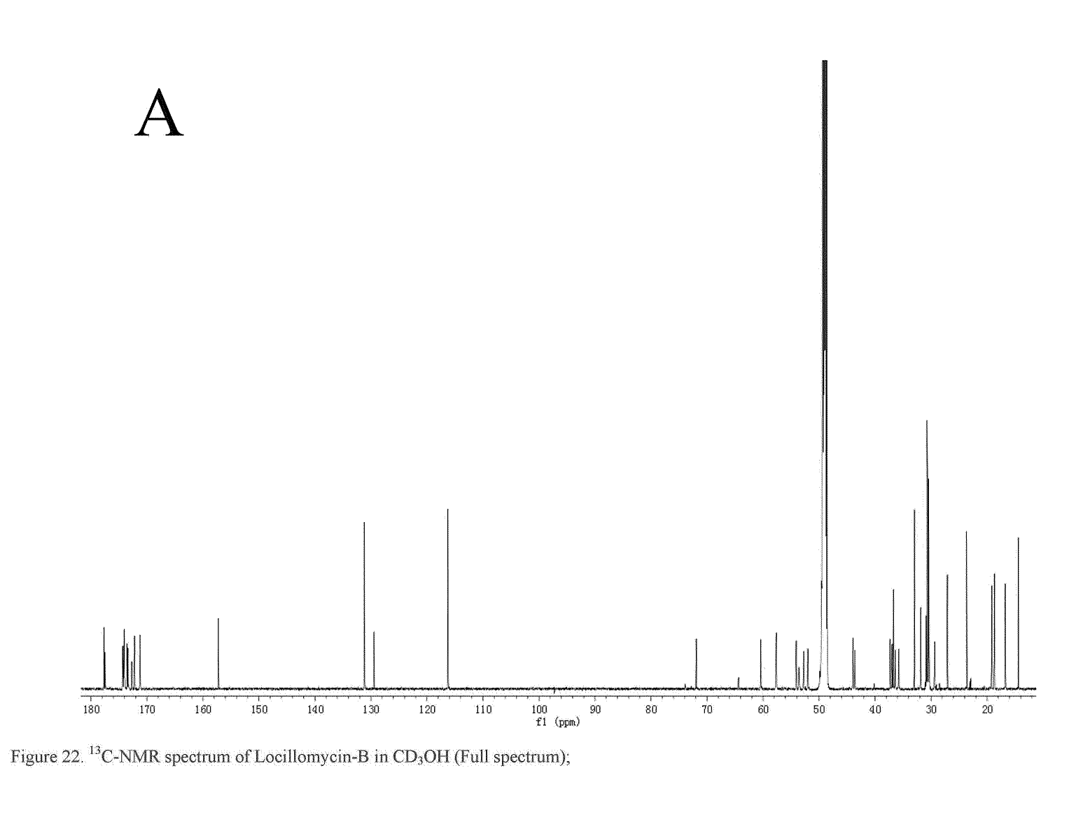

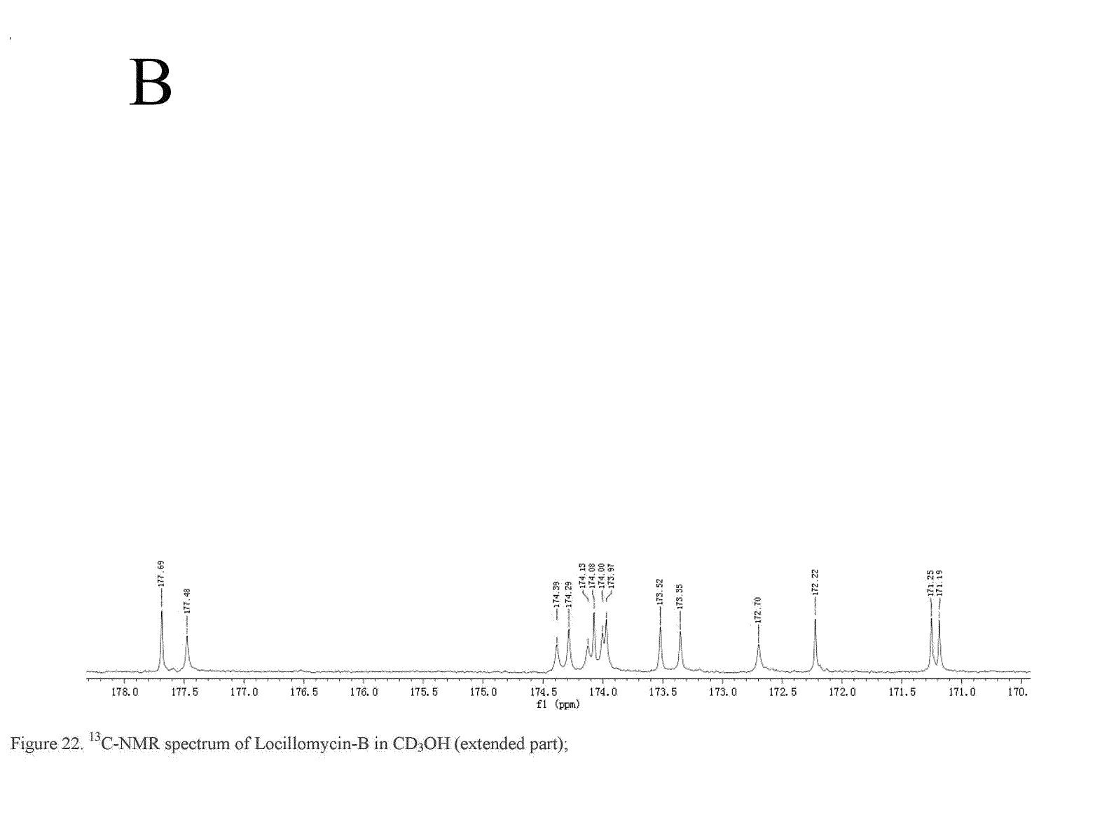

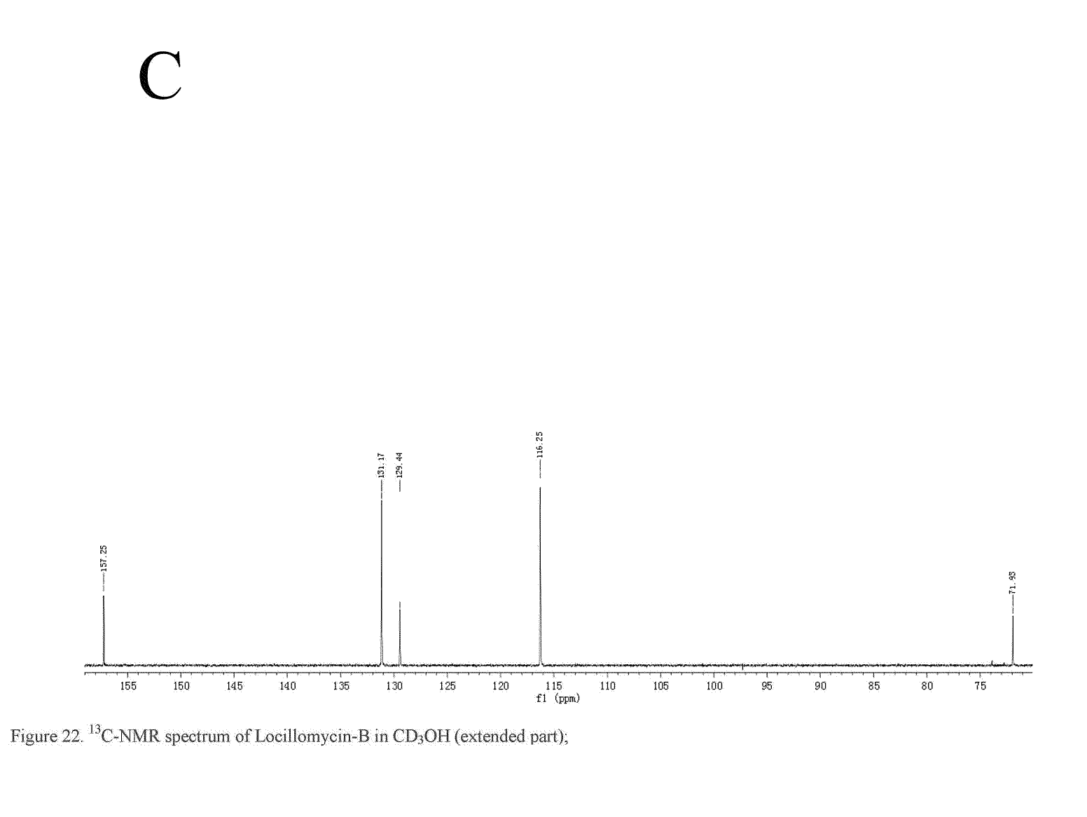

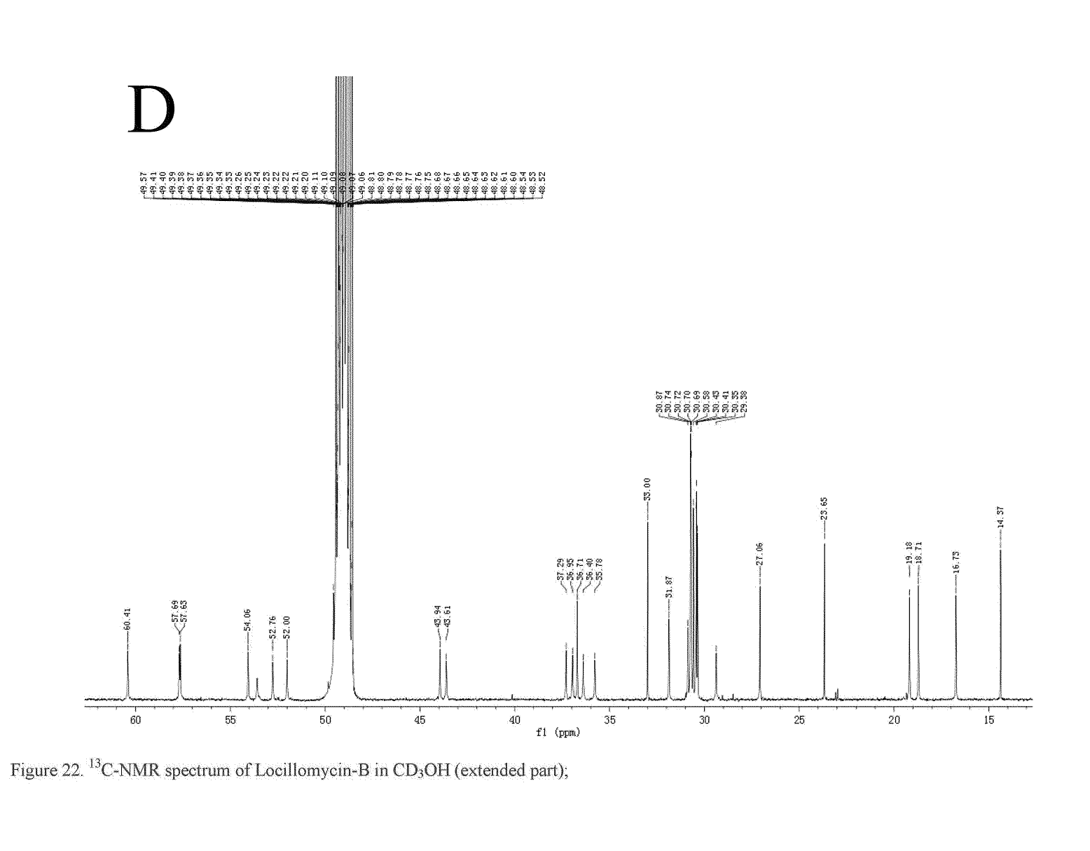

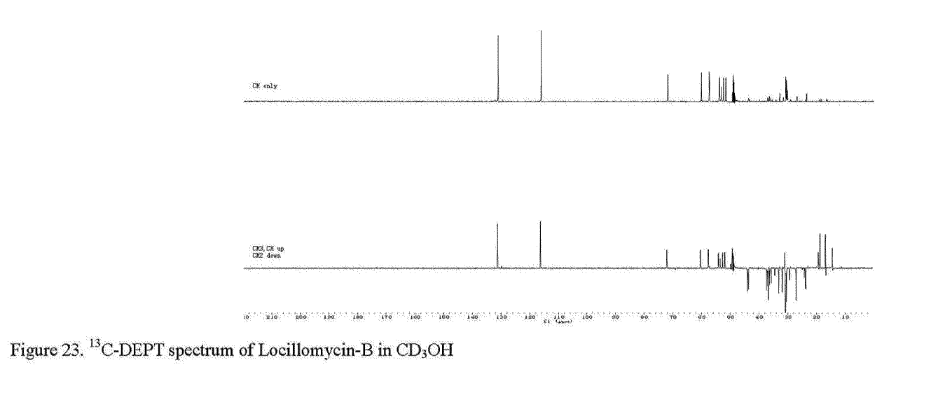

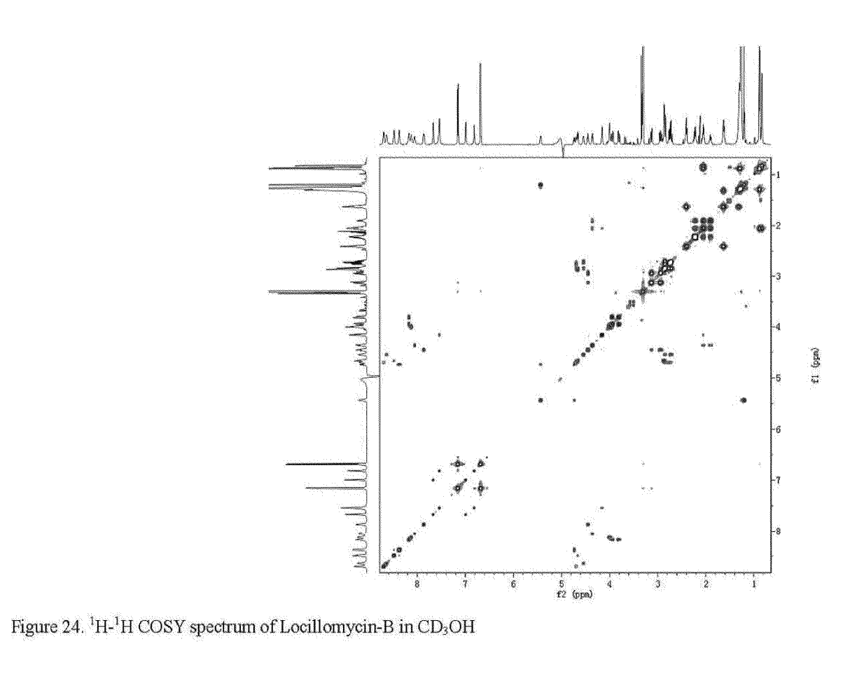

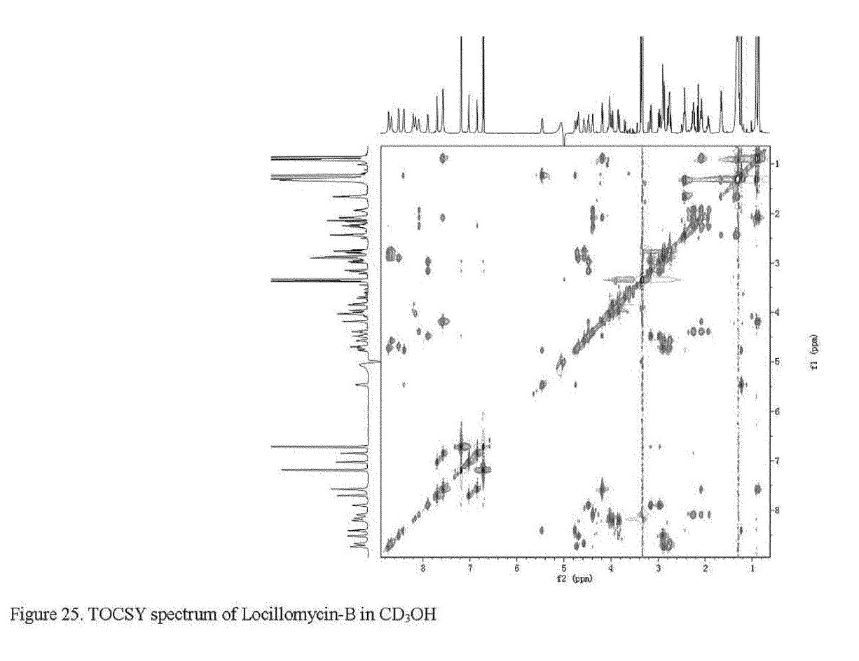

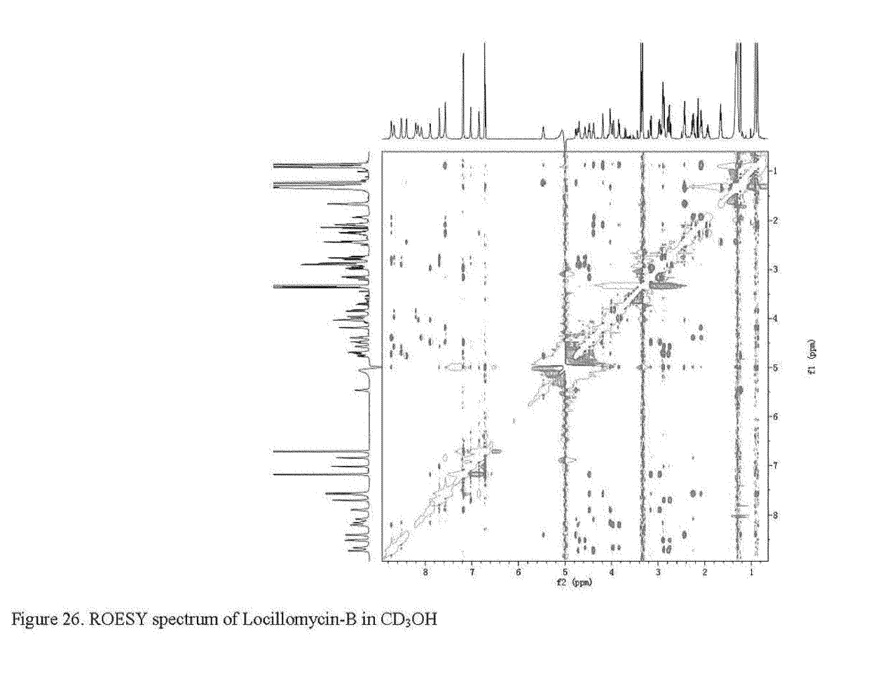

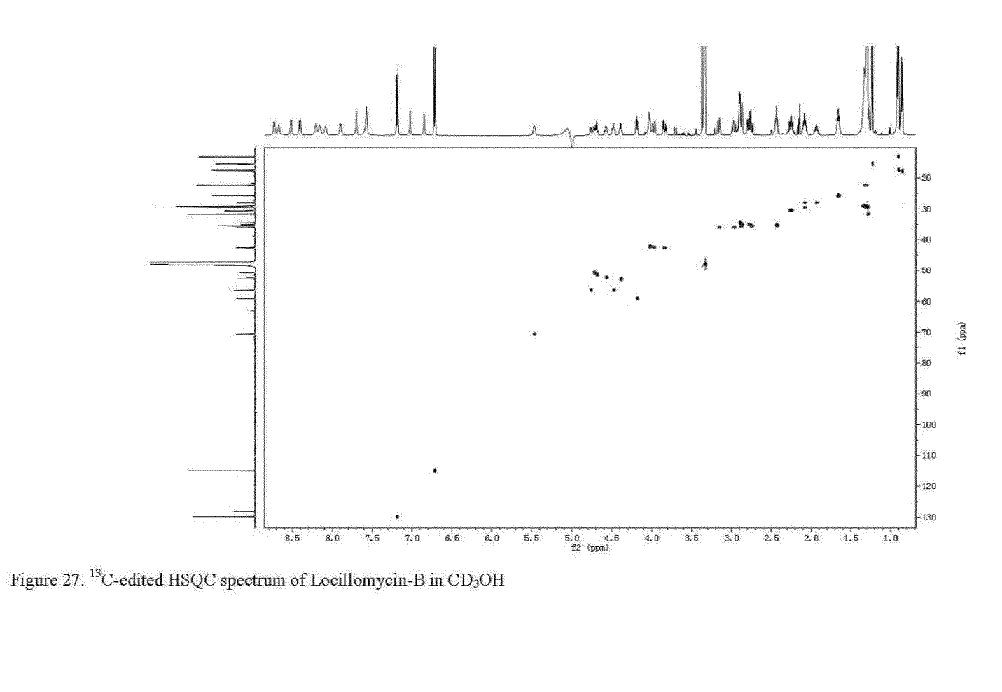

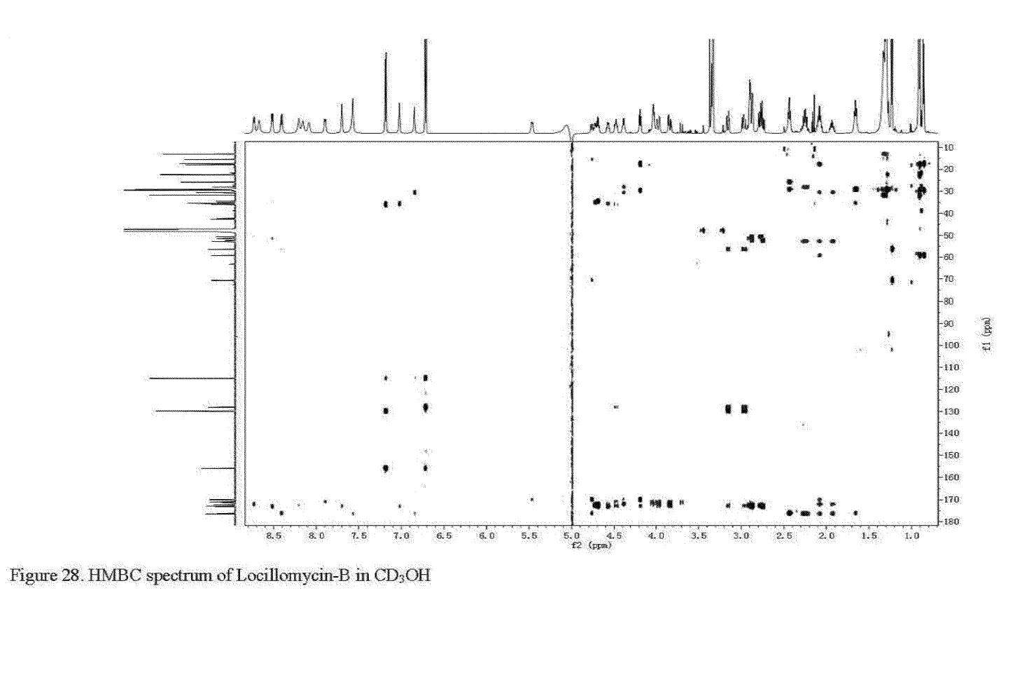

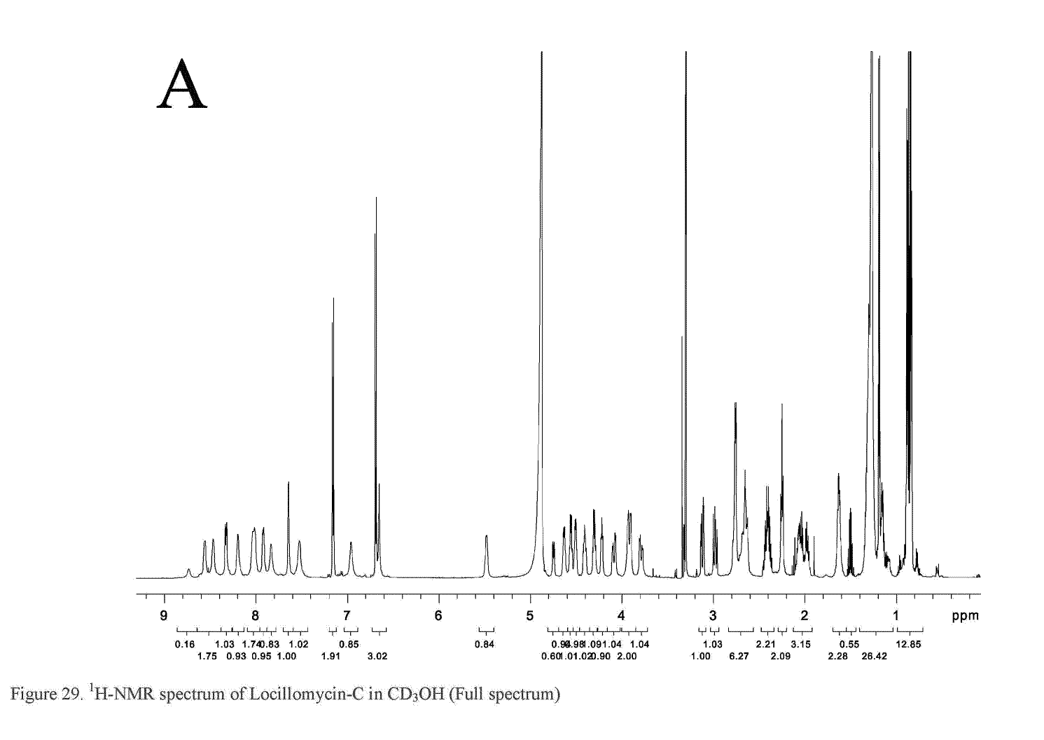

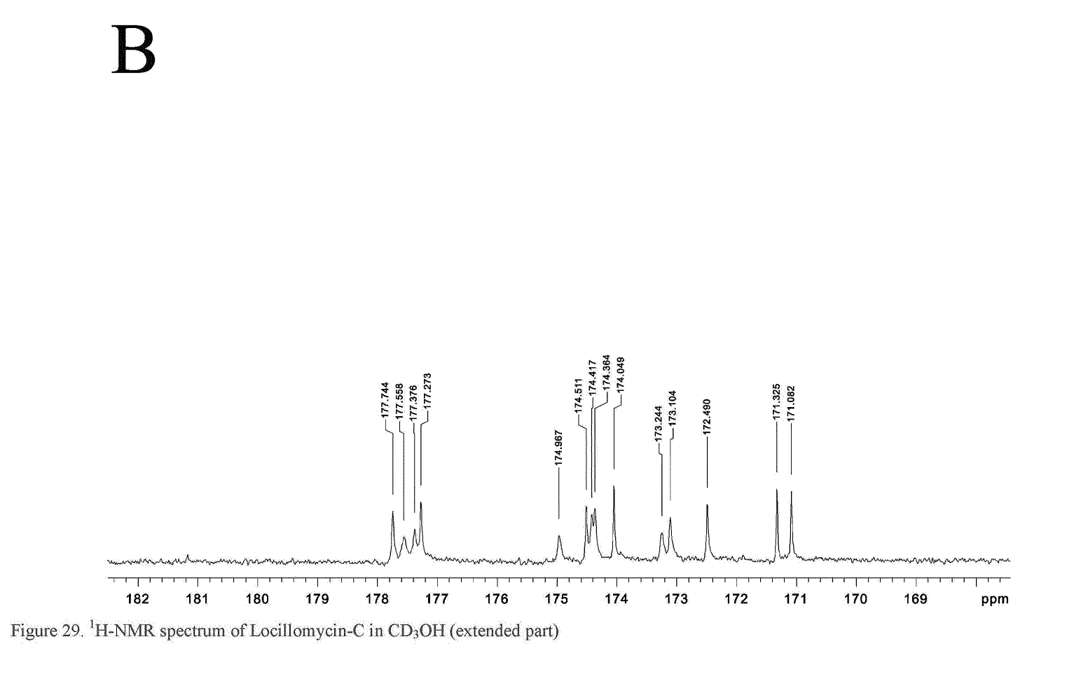

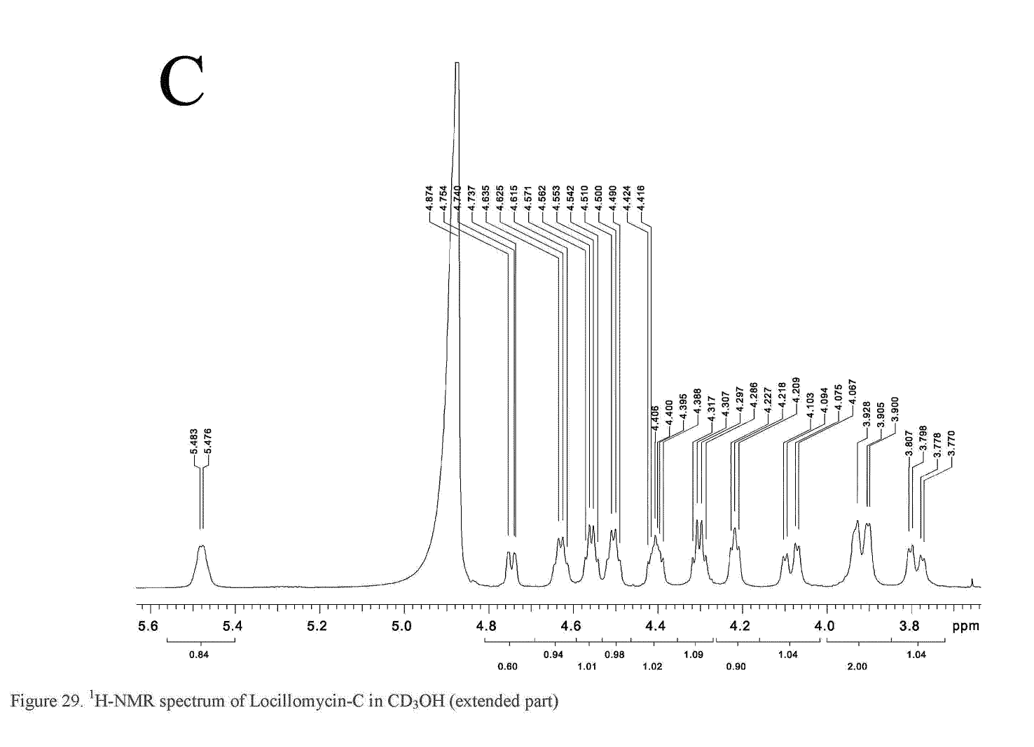

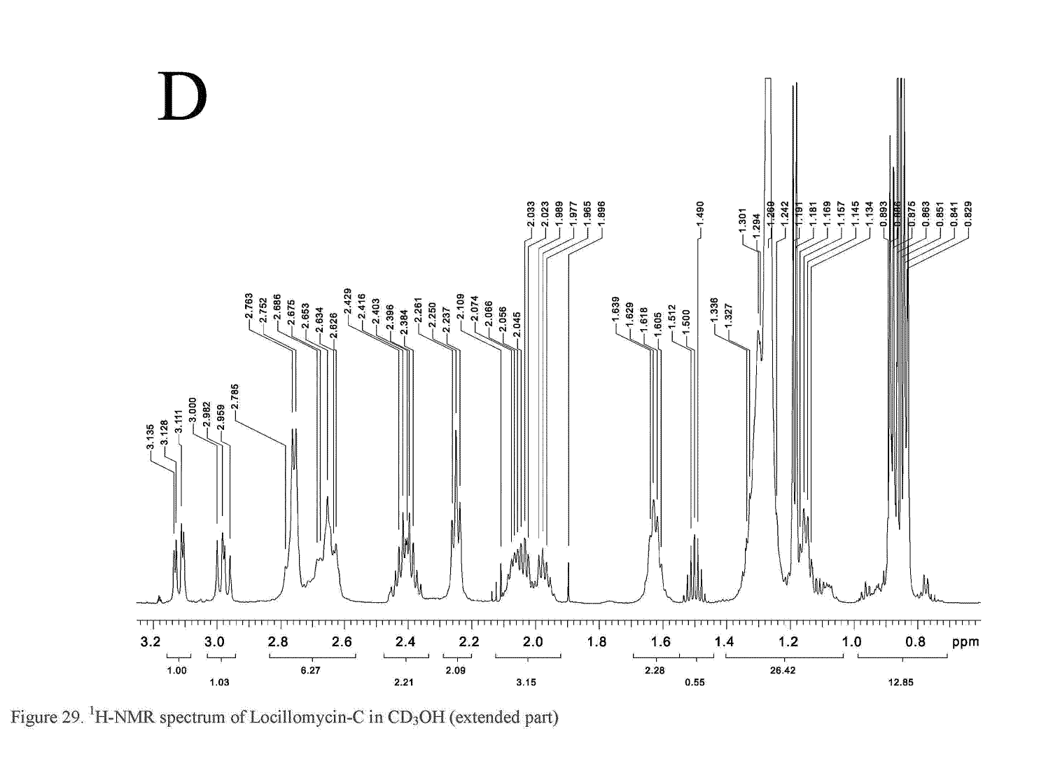

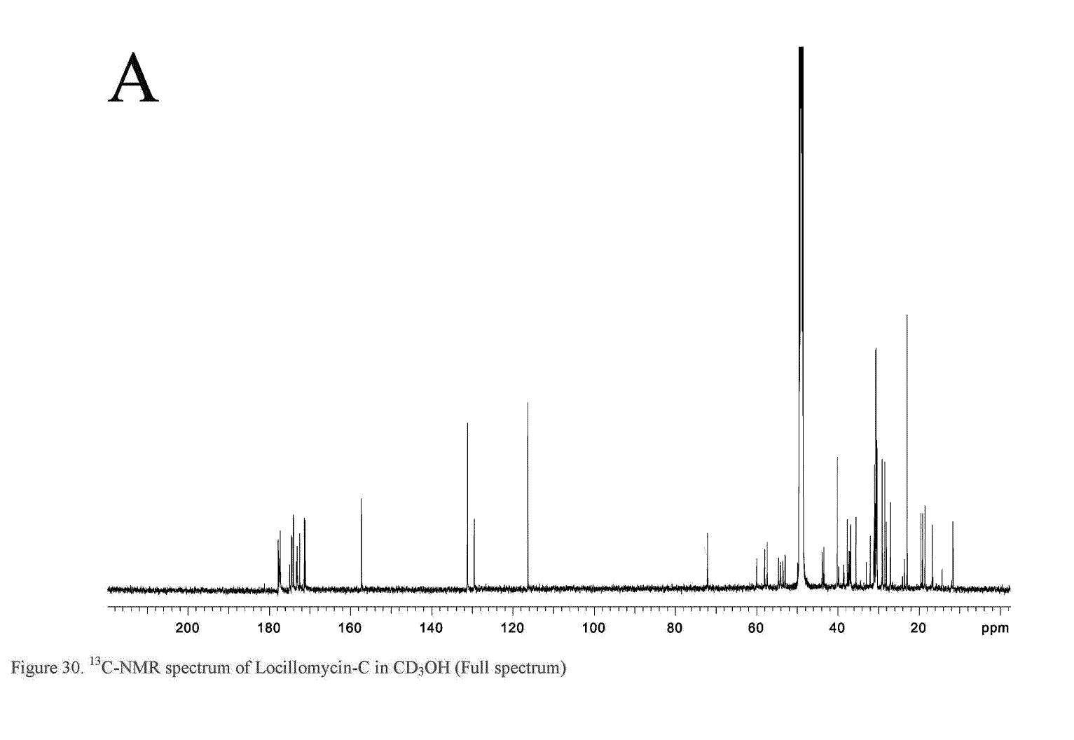

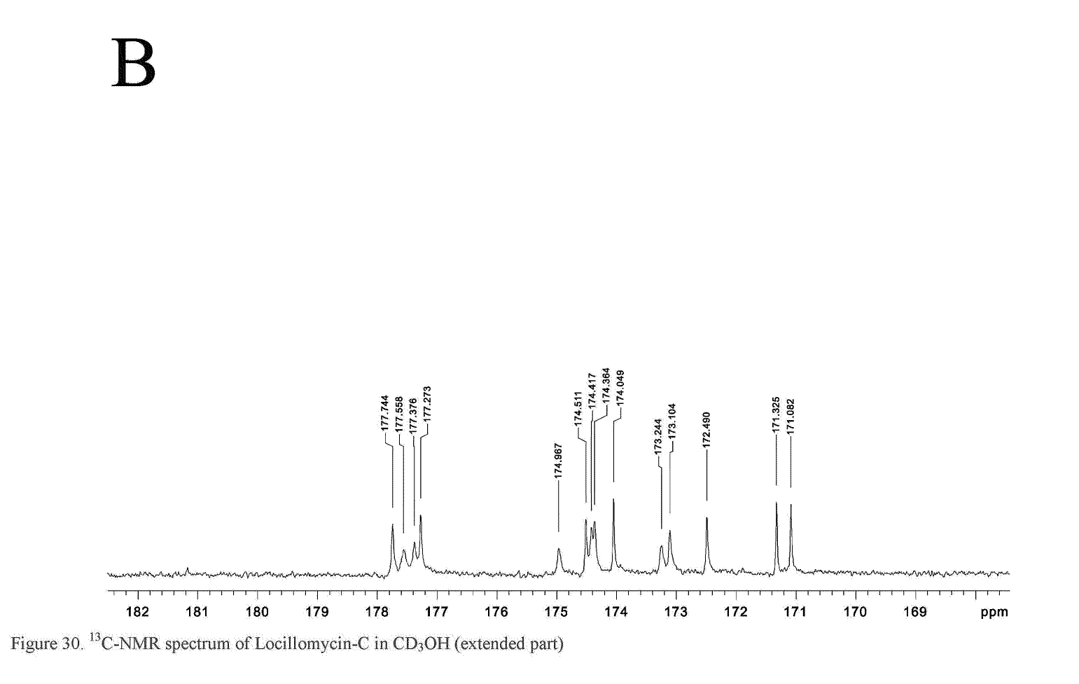

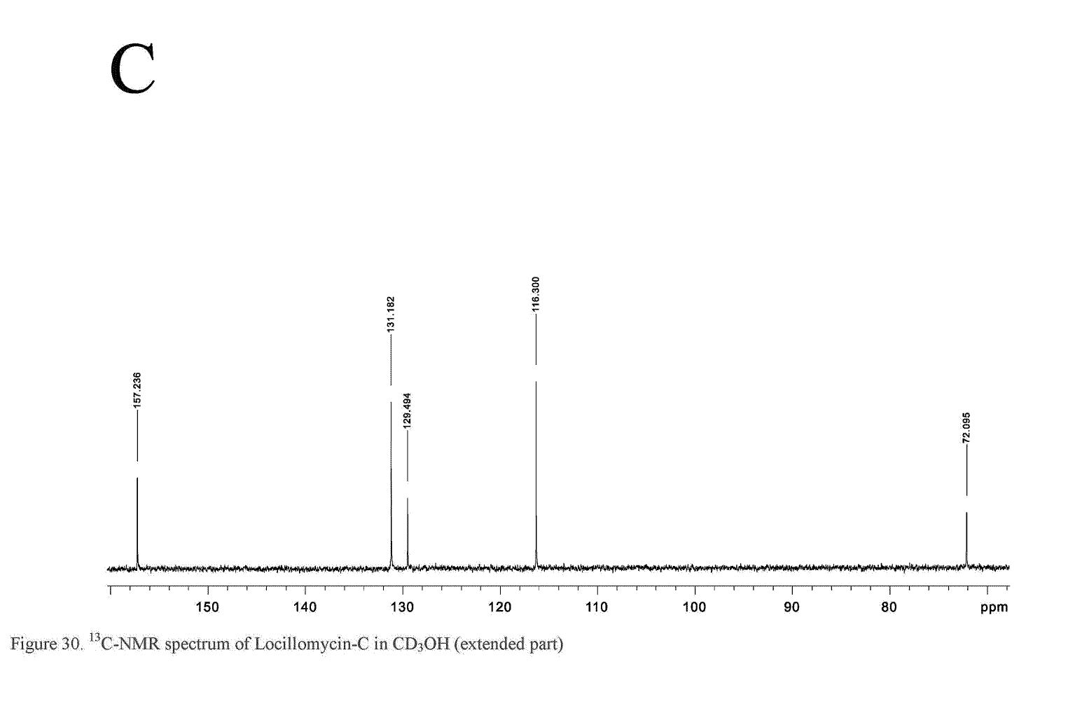

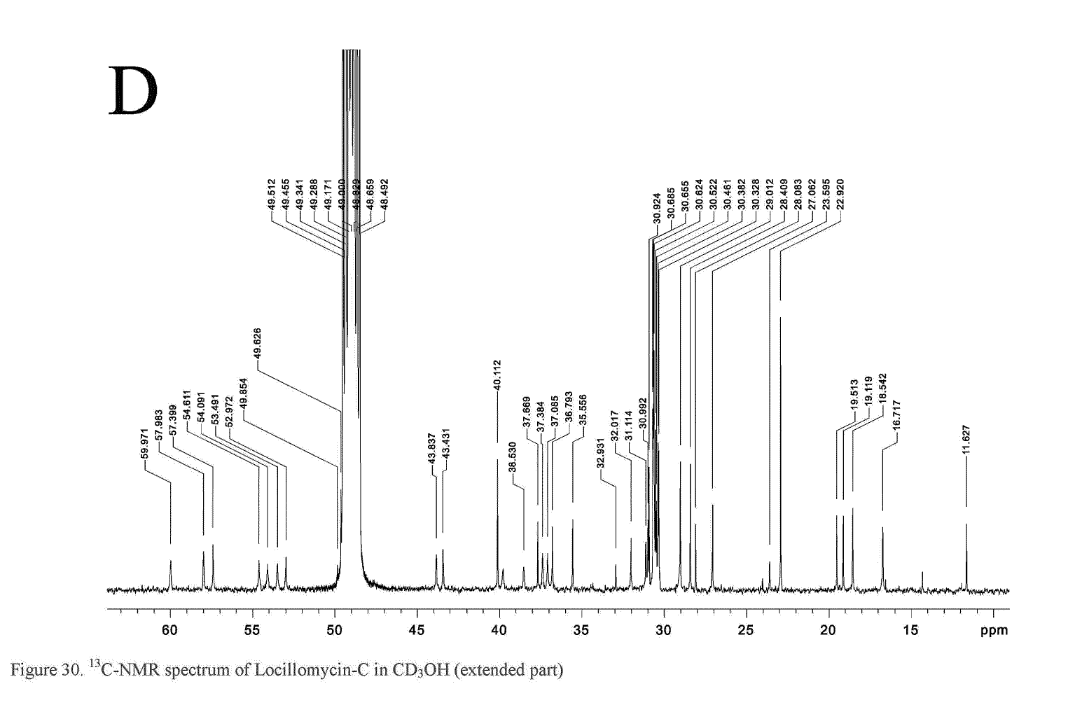

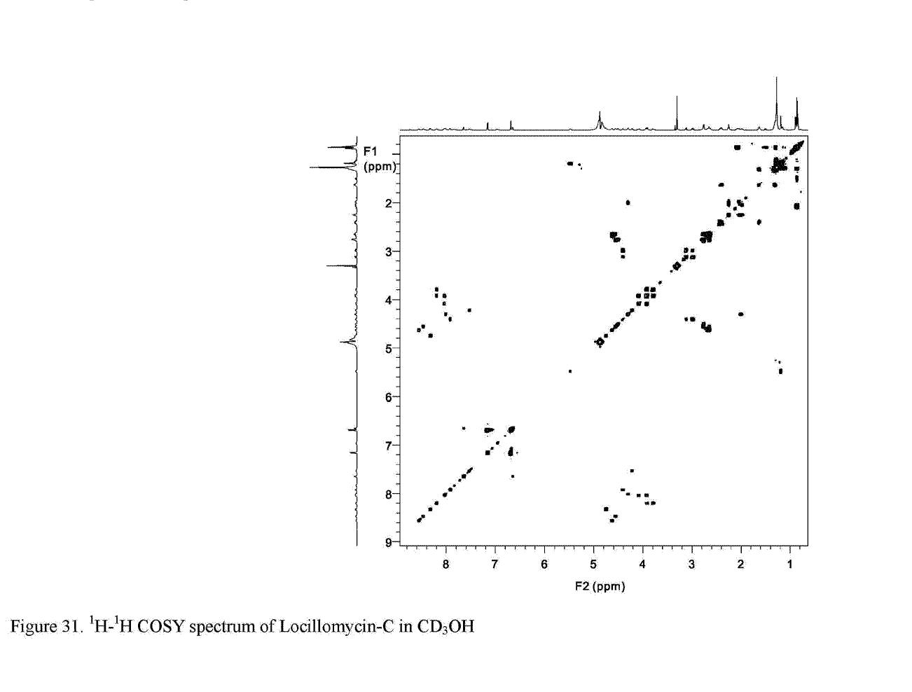

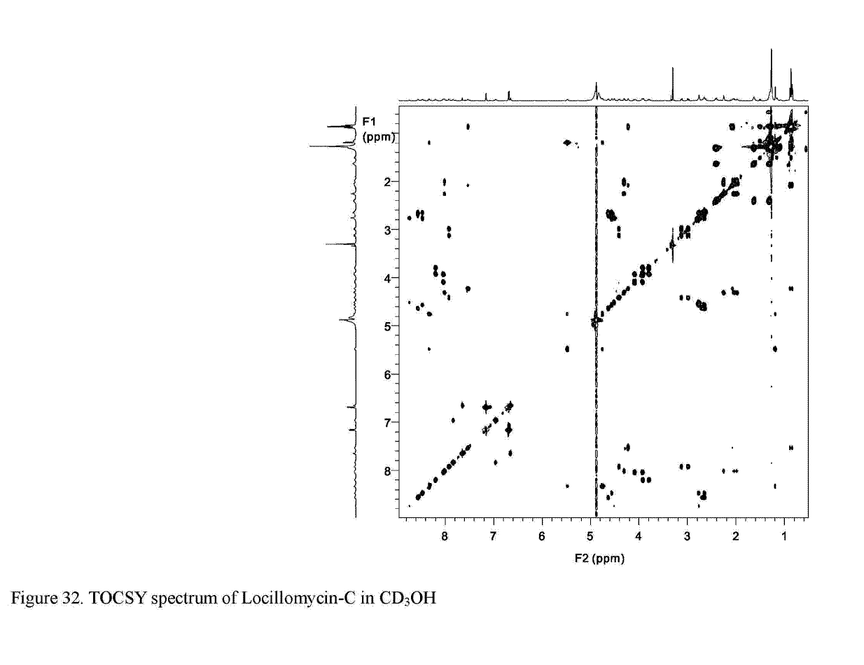

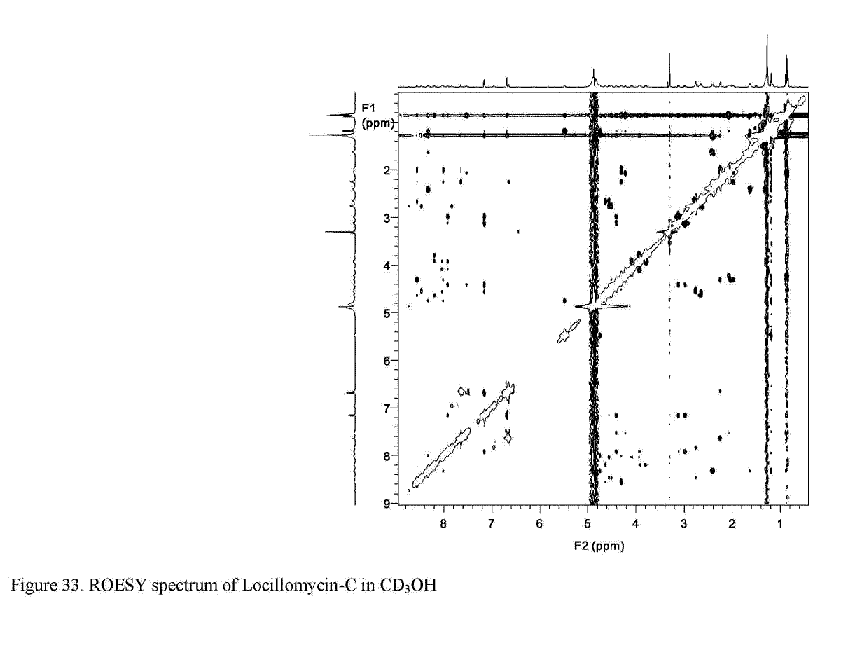

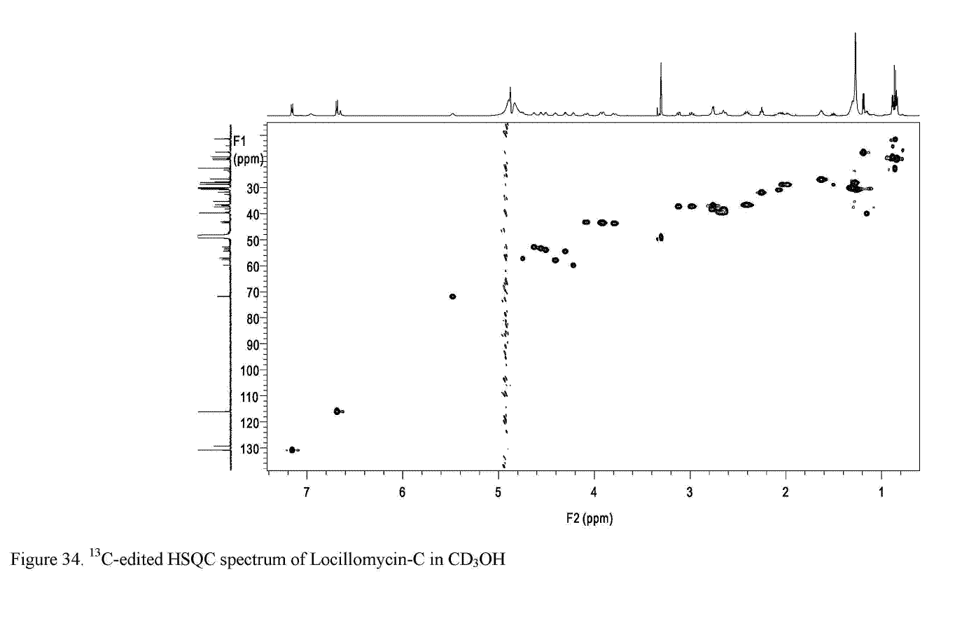

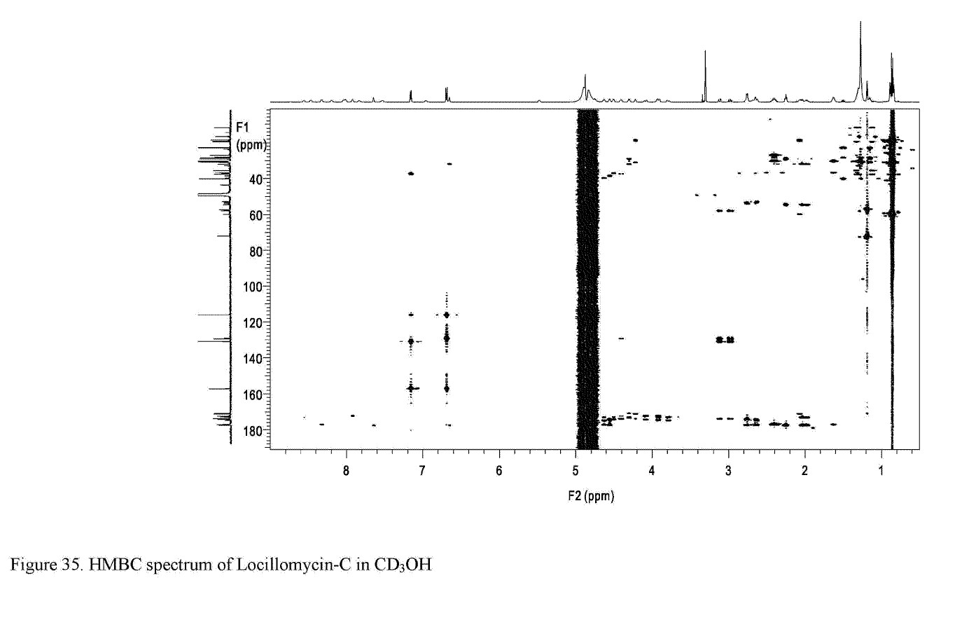

As for Locillomycin-A and Locillomycin-B, analysis of NMR and MS spectra showed that they have an identical core cyclic peptide as to the Locillomycin-C (see the attached FIGS. 1, 2, 3, 5, 6, 8, 9, 11, 12, 14, 15, and 17 to 28), except for a different molecular weight by 14 Dalton between each of the derivatives [Locillomycin-A (C₁₃H₂₅NO)<Locillomycin-B (C₁₄H₂₇NO)<Locillomycin-C(C₁₅H₂₉NO)], which comes from the different length of the long-chain acyl group. And their determination process will not be detailed here in this invention.

The NMR data of Locillomycin-A in H₂O/D₂O (90%/10%) is shown in Table 2.

TABLE 2
NMR data of Locillomycin-A (in 90% H₂O/10% D₂O)

| | | | | | |
|---|---|---|---|---|---|
| | | | | | C′ |
| residue | α (ppm) | β (ppm) | h_{N} (ppm) | others (ppm) | (ppm) |

Cyclic peptide (Chemical shifts of protons with
corresponding carbon chemical shifts in the parentheses)

| | | | | | |
|---|---|---|---|---|---|
| T1 | N/A (N/A) | 5.50 (74.34) | 8.34 | γ: 1.15 (18.78) | N/A |
| Q2 | 4.36 (55.73) | 1.91, 2.05 (30.52) | 8.03 | γ: 2.25 (33.53); Cδ: — (N/A); | N/A |
| | | | | ε: 6.73, 7.50 | |
| D3 | 4.64 (N/A) | 2.62, 2.69 (41.28) | 8.62 | Cγ: — (N/A) | N/A |
| G4 | 3.90, 3.95 (45.53) | — | 8.35 | — | N/A |
| N5 | 4.54 (55.18) | 2.79 (38.68) | 8.63 | Cγ: — (N/A); δ: 6.93, 7.68 | N/A |
| D6 | 4.64 (N/A) | 2.60, 2.72 (40.73) | 8.51 | Cγ: — (N/A) | N/A |
| G7 | 3.90, 3.99 (45.06) | — | 7.89 | — | N/A |
| Y8 | 4.52 (58.44) | 2.93, 3.08 (39.00) | 7.82 | Cγ: — (N/A); δ: 7.11 (N/A); | N/A |
| | | | | ε: 6.79 (N/A); Cζ: — (N/A) | |
| V9 | 4.23 (61.46) | 1.99 (33.07) | 7.76 | γ: 0.77 (20.22), 0.75 (20.71) | N/A |

long-chain acyl group (Chemical shifts of protons with
corresponding carbon chemical shifts in the parentheses, in ppm)

| | |
|---|---|
| 1′ | — (N/A) |
| 2′ | 2.49 (38.37) |
| 3′ | 1.62 (28.16) |
| 4′-11′ | 0.98~1.20 (30.7-32.7) |
| 12′ | N/A (N/A) |
| 13′ | 0.77 (13.69) |

note:
—: no such nucleus; N/A: assignment not available

The NMR data of Locillomycin-B in CD₃OH is shown in Table 3 (the chemical shifts of CD₃OH are taken as references, ¹H, 3.3 ppm, ¹³C: 49 ppm),

TABLE 3
The NMR data of Locillomycin-B (in CD₃OH)

| | | | | | |
|---|---|---|---|---|---|
| | | | H_{N} | | C′ |
| [symbol] | α (ppm) | β (ppm) | (ppm) | others (ppm) | (ppm) |

Cyclic peptide (Chemical shifts of protons with corresponding carbon
chemical shifts in the parentheses)

| | | | | | |
|---|---|---|---|---|---|
| T1 | 4.75 (57.63) | 5.44 (71.93) | 8.38 | γ: 1.21 (16.73) | 171.26 |
| Q2 | 4.36 (54.06) | 1.91, 2.06 (29.38) | 8.06 | γ: 2.23 (31.87); Cδ: — (177.69); | 173.43 |
| | | | | ε: 6.82, 5.54 | |
| D3 | 4.71 (52.00) | 2.77, 2.86 (36.40) | 8.70 | Cγ: — (174.13) | 173.97 |
| G4 | 3.93, 3.83 (43.94) | — | 8.17 | — | 172.70 |
| N5 | 4.55 (53.58) | 2.74, 2.84 (36.95) | 8.64 | Cγ: — (174.29); δ: 6.99, 7.67 | 174.39 |
| D6 | 4.67 (52.76) | 2.89 (35.78) | 8.49 | Cγ: — (174.00) | 173.52 |
| G7 | 3.96 (43.61) | — | 8.13 | — | 172.22 |
| Y8 | 4.47 (57.69) | 2.94, 3.14 (37.29) | 7.87 | Cγ: — (129.49); δ: 7.16 (131.17); | 174.08 |
| | | | | ε: 6.70 (116.25); Cζ: — (157.25) | |
| V9 | 4.17 (60.41) | 2.07 (30.87) | 7.57 | γ: 0.84 (19.18), 0.89 (18.71) | 171.44 |

long-chain acyl group (Chemical shifts of protons with corresponding
carbon chemical shifts in the parentheses, in ppm)

| | | |
|---|---|---|
| 1′ | — | (177.48) |
| 2′ | 2.41 | (36.81) |
| 3′ | 1.64 | (27.14) |
| 4′-12′ | 1.23~1.35 | (30.3-31.3) |
| 13′ | 1.30 | (23.75) |
| 14′ | 0.89 | (14.47) |

note:
—: no such nucleus;
N/A: assignment not available

The NMR data of Locillomycin-C in CD₃OH is shown in Table 3 (the chemical shifts of CD₃OH are taken as references, ¹H, 3.3 ppm, ¹³C: 49 ppm),

TABLE 4
The NMR data of Locillomycin-C (in CD₃OH)

| | | | | | |
|---|---|---|---|---|---|
| | | | H_{N} | | C′ |
| [symbol] | α (ppm) | β (ppm) | (ppm) | others (ppm) | (ppm) |

Cyclic peptide
(Chemical shifts of protons with corresponding carbon chemical shifts in the parentheses)

| | | | | | |
|---|---|---|---|---|---|
| T1 | 4.75 (57.43) | 5.48 (72.14) | 8.33 | γ: 1.19 (16.79) | 171.08 |
| Q2 | 4.30 (54.68) | 1.98, 2.04 (29.09) | 8.01 | γ: 2.25 (32.07); Cδ: — (177.74); | 173.24 |
| | | | | ε: 6.65, 7.64 | |
| D3 | 4.63 (53.04) | 2.65, 2.69 (39.81) | 8.56 | Cγ: — (177.37) | 174.97 |
| G4 | 3.92, 3.80 (43.90) | — | 8.20 | — | 173.10 |
| N5 | 4.51 (54.14) | 2.76 (37.14) | 8.74 | Cγ: — (174.41); δ: 6.96, 7.83 | 174.36 |
| D6 | 4.56 (53.53) | 2.65, 2.76 (38.57) | 8.47 | Cγ: — (177.55) | 174.51 |
| G7 | 4.09, 3.93 (43.46) | — | 8.04 | — | 172.49 |
| Y8 | 4.41 (58.05) | 2.98, 3.12 (37.43) | 7.92 | Cγ: — (129.49); δ: 7.16 (131.23); | 174.05 |
| | | | | ε: 6.69 (116.35); Cζ: — (157.24) | |
| V9 | 4.22 (59.99) | 2.07 (31.19) | 7.53 | γ: 0.84 (19.176), 0.88 (18.61) | 171.32 |

long-chain acyl group
(Chemical shifts of protons with corresponding carbon chemical shifts in the parentheses, in ppm)

| | | |
|---|---|---|
| 1′ | — | (177.27) |
| 2′ | 2.41 | (36.84) |
| 3′ | 1.63 | (27.14) |
| 4′-13′ | 1.2~1.4 | (30.3-31.0) |
| 14′ | 1.28 | (23.69) |
| 15′ | 0.86 | (11.71) |

note:
—: no such nucleus;
N/A: assignment not available

The 2^{nd} degree MS characteristics of molecule mass, relative abundance, molecule formula and speculated sequence of fragment of Locillomycin-A are summarized in Table 5.

TABLE 5
The 2^{nd} degree MS characteristics
of the fragment of Locillomycin-A

| Molecule | Relative | Molecule | |
|---|---|---|---|
| Weight | Abundance | Formula | Sequence |
| 212.1 | 53 | C₁₃H₂₆O₁N₁ | CH₃(CH₂)₁₁CONH |
| 221.1 | 105 | C₁₁H₁₃N₂O₃ | Gly-Tyr |
| 244.1 | 74 | C₉H₁₄N₃O₅ | Gln-Asp |
| 280.3 | 268 | C₁₄H₂₂N₃O₃ | Tyr-Val |
| 297.1 | 51 | C₁₇H₃₃N₂O₂ | Thr(Link) |
| 301.3 | 50 | C₁₁H₁₇N₄O₆ | Gln-Asp-Gly |
| 336.3 | 60 | C₁₅H₁₈N₃O₆ | Asp-Gly-Tyr |
| 397.4 | 55 | C₂₂H₄₁N₂O₄ | Val-Thr(Link) |
| 398.2 | 63 | C₂₁H₄₀N₃O₄ | Thr(Link)-Gln |
| 402.1 | 54 | C₁₄H₂₀N₅O₉ | Asp-Gly-Asn-Asp |
| 415.4 | 81 | C₁₅H₂₃N₆O₈ | Gln-Asp-Gly-Asn |
| 459.2 | 47 | C₁₆H₂₃N₆O₁₀ | Asp-Gly-Asn-Asp-Gly |
| 507.1 | 72 | C₂₁H₂₇N₆O₉ | Gly-Asn-Asp-Gly-Thr |
| 530.2 | 277 | C₁₉H₂₈N₇O₁₁ | Gln-Asp-Gly-Asn-Asp |
| 587.1 | 106 | C₂₁H₃₁N₈O₁₂ | Gln-Asp-Gly-Asn-Asp- |
| | | | Gly |
| 622.2 | 97 | C₂₅H₃₂N₇O₁₂ | Asp-Gly-Asn-Asp-Gly- |
| | | | Tyr |
| 640.4 | 74 | C₃₁H₅₄N₅O₉ | Val-Thr(Link)-Gln-Asp |
| 688 | 69 | C₃₆H₅₈N₅O₈ | Tyr-Val-Thr(Link)-Gln |
| 721.2 | 83 | C₃₀H₄₁N₈O₁₃ | Asp-Gly-Asn-Asp-Gly- |
| | | | Tyr-Val |
| 732.1 | 49 | C₃₇H₅₈N₅O₁₀ | Asp-Gly-Tyr-Val- |
| | | | Thr(Link) |
| 750.3 | 51 | C₃₀H₄₀N₉O₁₄ | Gln-Asp-Gly-Asn-Asp- |
| | | | Gly-Thr |
| 849.4 | 193 | C₃₅H₄₉N₁₀O₁₅ | Gln-Asp-Gly-Asn-Asp- |
| | | | Gly-Tyr-Val |
| 860.5 | 52 | C₄₂H₆₆N₇O₁₂ | Tyr-Val-Thr(Link)- |
| | | | Gln-Asp-Gly |
| 884.4 | 50 | C₃₈H₆₂N₉O₁₅ | Thr(Link)-Gln-Asp- |
| | | | Gly-Asn-Asp-G1y3 |
| 975.6 | 41 | C₄₆H₇₁N₈O₁₅ | Asp-Gly-Tyr-Val-Thr |
| | | | (Link)-Gln-Asp |
| 983.6 | 47 | C₄₃H₇₁N₁₀O₁₆ | Val-Thr(Link)-Gln- |
| | | | Asp-Gly-Asn-Asp |
| 1146.6 | 404 | C₅₂H₈₀N₁₁O₁₈ | locillomycin A + H |

Thr(link): represent the amino of a Thr reacted with the fatty acid (CH₃(CH₂)₁₁COOH) to form amide bond. The 2″ degree MS characteristics of molecule mass, relative abundance, molecule formula and speculated sequence of fragment of Locillomycin-B are summarized in Table 6.

TABLE 6
The 2^{nd} degree MS characteristics of
the fragment of Locillomycin-B

| Molecule | Relative | Molecule | |
|---|---|---|---|
| Weight | Abundance | Formula | Sequence |
| 221 | 47 | C₁₁H₁₃N₂O₃ | Gly-Tyr |
| 244 | 87 | C₉H₁₄N₃O₅ | Gln-Asp |
| 287.1 | 48 | C₁₀H₁₅N₄O₆ | Asn-Asp-Gly |
| 336.1 | 81 | C₁₅H₁₈N₃O₆ | Asp-Gly-Tyr |
| 402.4 | 56 | C₁₄H₂₀N₅O₉ | Asp-Gly-Tyr-Asp |
| 415 | 54 | C₁₅H₂₃N₆O₈ | Gln-Asp-Gly-Asn |
| 507.3 | 61 | C₂₁H₂₇N₆O₉ | Gly-Asn-Asp-Gly-Tyr |
| 587.2 | 99 | C₂₁H₃₁N₈O₁₂ | Gln-Asp-Gly-Asn-Asp-Gly |
| 603.9 | 76 | C₃₃H₅₅N₄O₆ | Gly-Tyr-Val-Thr(Link) |
| 622 | 130 | C₂₅H₃₂N₇O₁₂ | Asp-Gly-Asn-Asp-Gly-Tyr |
| 721.2 | 94 | C₃₀H₄₁N₈O₁₃ | Asp-Gly-Asn-Asp-Gly- |
| | | | Tyr-Val |
| 750.1 | 45 | C₃₀H₄₀N₉O₁₄ | Gln-Asp-Gly-Asn-Asp- |
| | | | Gly-Tyr |
| 832.1 | 63 | C₄₁H₆₆N₇O₁₁ | Asn-Asp-Gly-Tyr-Val- |
| | | | Thr(Link) |
| 849.3 | 233 | C₃₅H₄₉N₁₀O₁₅ | Gln-Asp-Gly-Asn-Asp- |
| | | | Gly-Tyr-Val |
| 898.7 | 89 | C₃₉H₆₄N₉O₁₅ | Thr(Link)-Gln-Asp-Gly- |
| | | | Asn-Asp-Gly |
| 917.5 | 80 | C₄₄H₆₉N₈O₁₃ | Gly-Asn-Asp-Gly-Tyr- |
| | | | Val-Thr(Link) |
| 940.1 | 50 | C₄₂H₇₀N₉O₁₅ | Val-Thr(Link)-Gln-Asp- |
| | | | Gly-Asn-Asp |
| 1160.6 | 366 | C₅₃H₈₂N₁₁O₁₈ | locillomycin B + H |

Thr(link): represent the amino of a Thr reacted with the fatty acid (CH₃(CH₂)₁₂COOH) to form amide bond. The 2^{nd} degree MS characteristics of molecule mass, relative abundance, molecule formula and speculated sequence of fragment of Locillomycin-C are summarized in Table 7.

TABLE 7
The 2nd degree MS characteristics of the fragment of Locillomycin-C

| Molecule | Relative | Molecule | |
|---|---|---|---|
| Weight | Abundance | Formula | Sequence |
| 172.2 | 62 | C₆H₁₀N₃O₃ | Gly-Asn |
| 221 | 190 | C₁₁H₁₃N₂O₃ | Gly-Tyr |
| 230.1 | 49 | C₈H₁₂N₃O₅ | Asn-Asp |
| 244 | 303 | C₉H₁₄N₃O₅ | Gln-Asp |
| 287.1 | 181 | C₁₀H₁₅N₄O₆ | Asn-Asp-Gly |
| 301.2 | 106 | C₁₁H₁₇N₄O₆ | Gln-Asp-Gly |
| 326 | 87 | C₁₉H₃₆N₁O₃ | Thr(Link) |
| 336 | 94 | C₁₅H₁₈N₃O₆ | Asp-Gly-Tyr |
| 344.1 | 50 | C₁₂H₁₈N₅O₇ | Gly-Asn-Asp-Gly |
| 402.2 | 69 | C₁₄H₂₀N₅O₉ | Asp-Gly-Asn-Asp |
| 415.2 | 100 | C₁₅H₂₃N₆O₈ | Gln-Asp-Gly-Asn |
| 425.2 | 79 | C₂₄H₄₅N₂O₄ | Val-Thr(Link) |
| 450.1 | 65 | C₁₉H₂₄N₅O₈ | Asn-Asp-Gly-Tyr |
| 459.1 | 51 | C₁₆H₂₃N₆O₁₀ | Asp-Gly-Asn-Asp |
| 507.1 | 59 | C₂₁H₂₇N₆O₉ | Gly-Asn-Asp-Gly-Tyr |
| 530 | 124 | C₁₉H₂₈N₇O₁₁ | Gln-Asp-Gly-Asn-Asp |
| 553 | 192 | C₂₉H₅₃N₄O₆ | Val-Thr(Link)-Gln |
| 569.3 | 99 | C₂₈H₄₉N₄O₈ | Thr(Link)-Gln-Asp |
| 587 | 79 | C₂₁H₃₁N₈O₁₂ | Gln-Asp-Gly-Asn-Asp-Gly |
| 588.4 | 72 | C₃₃H₅₄N₃O₆ | Tyr-Val-Thr(Link) |
| 622.1 | 72 | C₂₅H₃₂N₇O₁₂ | Asp-Gly-Asn-Asp-Gly |
| 716.1 | 52 | C₃₈H₆₂N₅O₈ | Tyr-Val-Thr(Link)-Gln |
| 849.2 | 165 | C₃₅H₄₉N₁₀O₁₅ | Gln-Asp-Gly-Asn-Asp-Gly-Tyr-Val |
| 912.2 | 61 | C₄₀H₆₆N₉O₁₅ | Thr(Link)-Gln-Asp-Gly-Asn-Asp-Gly |
| 931.8 | 60 | C₄₅H₇₁N₈O₁₃ | Gly-Asn-Asp-Gly-Tyr-Val-Thr(Link) |
| 1174.6 | 69 | C₅₄H₈₄N₁₁O₁₈ | LocillomycinC + H |

Thr(link): represent the amino of a Thr reacted with the fatty acid (CH₃(CH₂)₁₃COOH) to form amide bond.

The HPLC analysis revealed the locillomycin derivatives have a core cyclic peptide with amino acid residues sequentially as L-Thr, D-Gln, L-Asp, L-Gly, L-Asn, L-Asp, L-Gly, L-Tyr and L-Val.

### Example 6

### Experiment on Anti-Fungus with Locillomycin-A, Locillomycin-B and Locillomycin-C

The anti-fungi experiment, with 6 treatments of different drug concentration and three replicates each treatment and sterile water as blank check, was conducted by mixing Locillomycin-A, Locillomycin-B and Locillomycin-C separately into different plates containing LB medium and, then, inoculating pathogenic fungi in the center of the plates, followed by incubation at 28° C. to check the effectiveness of antifungal function. The criteria of the effectiveness were defined as the size of orthogonal cross diameter of the pathogen growth. The experiments revealed that Locillomycin-A, Locillomycin-B and Locillomycin-C have strong antibiotic activities for inhibition of fungi (Table 8).

TABLE 8
The effects of Locillomycin-A, Locillomycin-B and Locillomycin-C
on Pathogenic fungi

| | Locillomycin A | | Locillomycin B | | Locillomycin C | |
|---|---|---|---|---|---|---|
| Pathogenic | IC₅₀ | IC₉₀ | IC₅₀ | IC₉₀ | IC₅₀ | IC₉₀ |
| Fungi | (μg/ml) | (μg/ml) | (μg/ml) | (μg/ml) | (μg/ml) | (μg/ml) |
| Rhizoctonia | 15.5 | 45.9 | 13.8 | 40.6 | 12.4 | 38.6 |
| solani | | | | | | |
| Fusarium | 12.5 | 56.3 | 11.6 | 48.9 | 10.3 | 46.8 |
| oxysporum | | | | | | |
| Fusarium | 25.4 | >100 | 22.6 | >100 | 21.7 | >100 |
| graminearum | | | | | | |

### Example 7

### Experiment on Anti-Bacterium with Locillomycin-A, Locillomycin-B and Locillomycin-C

The anti-bacterium experiment, with sterile water as blank check, was conducted by mixing different doses of Locillomycin-A, Locillomycin-B and Locillomycin-C separately into different plates containing LB medium and, then, evenly inoculating rice bacterial leaf spot pathogen onto the surface of the plates, followed by inverted incubation at 28° C. to check the effectiveness of antifungal function. The criteria of the effectiveness were defined as the minimum inhibition concentration (MIC) in the non-bacterium colony plate of the pathogen growth. The experiments revealed that the MIC of Locillomycin-A was 6.3 μg/ml; Locillomycin-B was 5.8 μg/ml; and Locillomycin-C was 5.4 μg/ml. The results demonstrated that Locillomycin-A, Locillomycin-B and Locillomycin-C have strong antibiotic activities for inhibition of bacterium growth.

### Example 8

### Experiment on Anti-Virus with Locillomycin-A, Locillomycin-B and Locillomycin-C

The anti-virus experiment was designed using Porcine Epidemic Diarrhea Virus (PEDV) as an indicator; conducted on a 24-well plate containing a single layer of Vero cell in each well, which was infected with PEDV at a multiplicity of infection (MOI), and incubated at 37° C. After one hour, different concentrations of mixtures of Locillomycin-A, Locillomycin-B and Locillomycin-C were introduced to treatments, and continuously incubated at 37° C. for 36 hours. At the end of the experiment, RNA of the virus was extracted from the wells, and quantified with fluorescent qPCR. The results revealed that the PEDV infection could be effectively inhibited by locillomycin mixture especially that of concentration at the 10 μg/ml, which reduced the virus copy by 300 times.

The invention claimed is:

1. Three new compounds of cyclic lipopeptide antibiotic Locillomycin-A, Locillomycin-B and Locillomycin-C thereof, according to structural formulae (I): 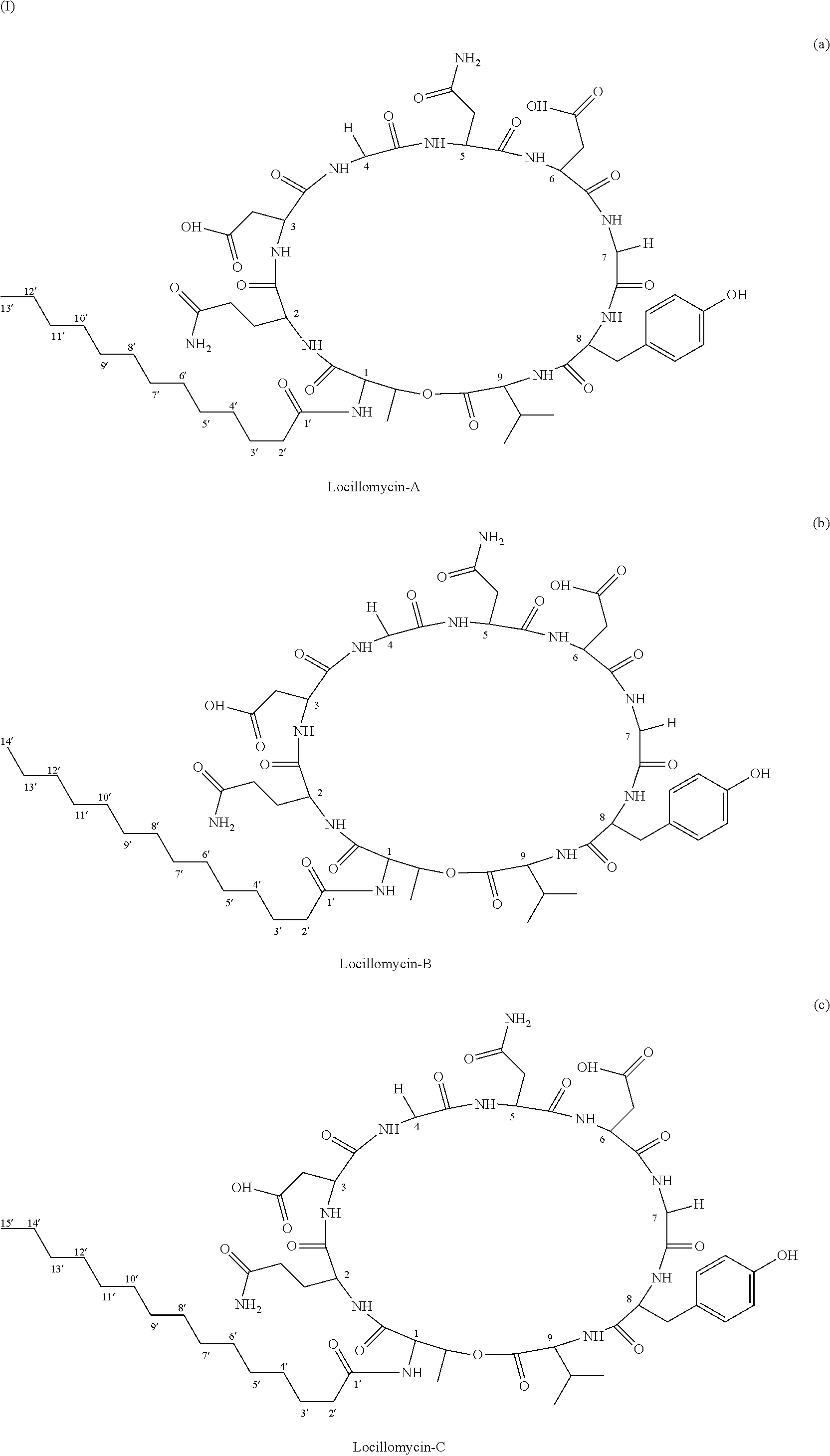 [SMILES: "(I)" ; CCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C ; CCCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C ; CCCCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C]
wherein:
1, or 2, or 3, or 4, or 5, or 6, or 7, or 8, or 9 is an amino acid residue thereof, 1: Thr; 2: Glu; 3, or 6: Asp; 4, or 7: Gly; 5: Asn; 8: Tyr; 9: Val; and
1′ to 13′, or 1′ to 14′, or 1′ to 15′ is a straight carbon chain wherein, the general chemical composition is CₙHₘNO, within which, the combinations of n and m can be one of the three: n=13 and m=25, or n=14 and m=27, or n=15 and m=29.

2. A method of making three new compounds of cyclic lipopeptide antibiotic Locillomycin-A, Locillomycin-B and Locillomycin-C thereof, according to structural formulae (I): 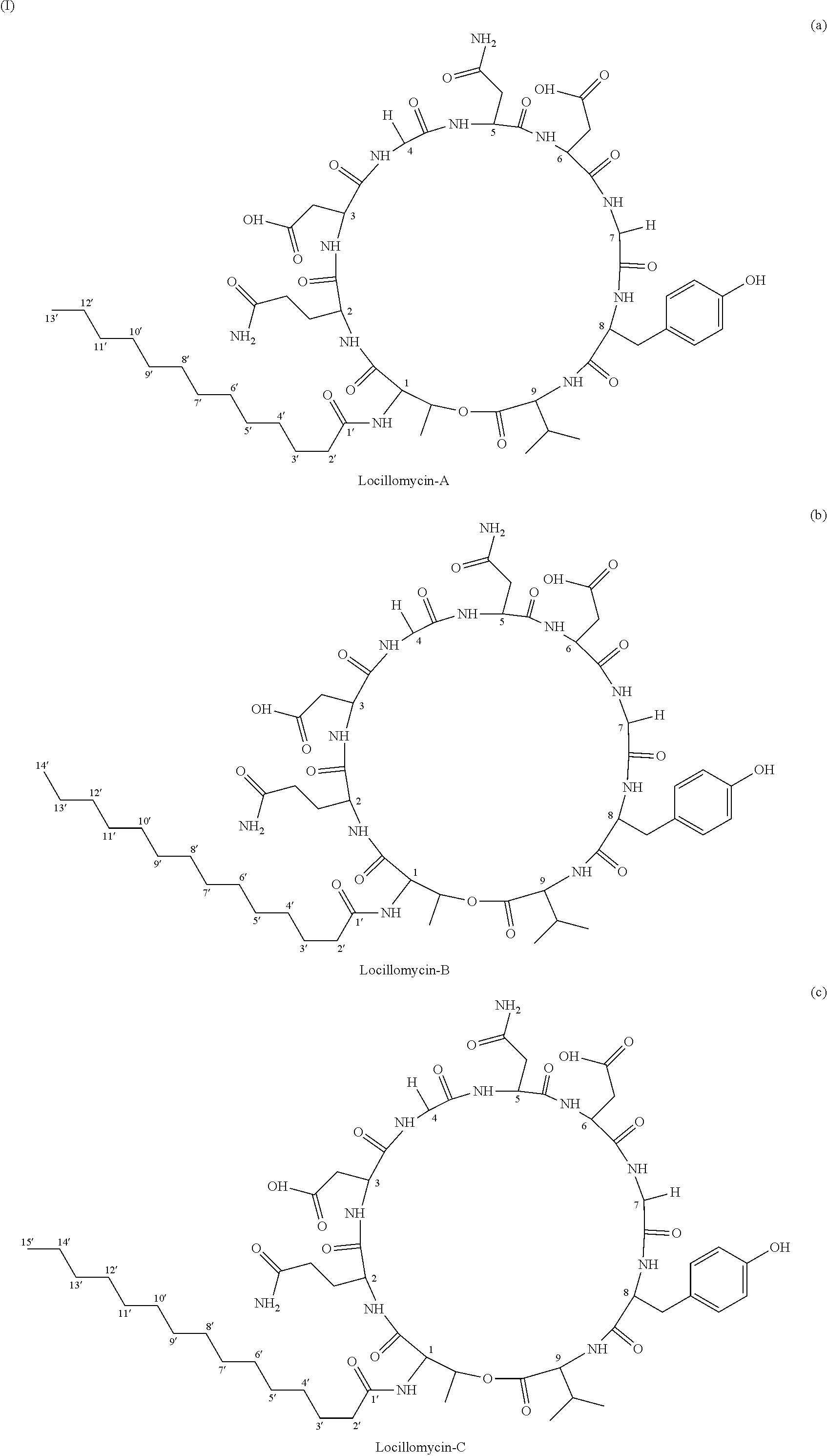
wherein:
1, or 2, or 3, or 4, or 5, or 6, or 7, or 8, or 9 is an amino acid residue thereof, 1: Thr; 2: Glu; 3, or 6: Asp; 4, or 7: Gly; 5: Asn; 8: Tyr; 9: Val; and 1′ to 13′, or 1′ to 14′, or 1′ to 15′ is a straight carbon chain wherein, the general chemical composition is CₙHₘNO, within which, the combinations of n and m can be one of the three: n=13 and m=25, or n=14 and m=27, or n=15 and m=29 characterized by fermentation of Bacillus subtilis Bs916 (CGMCC No. 0808: Classification number in China General Microbiological Culture Collection Center), and followed by a process of purification.

3. A method of making the compounds according to claim 2, wherein the fermentation is characterized by culturing Bacillus subtilis Bs916 in two stages: firstly, culturing Bs916 in tubes containing LB liquid medium at 37° C. and shaking at 200 r/m on a shaker for 24 hours; secondly, transferring the medium from the first stage into bottles containing LB liquid medium at 28° C. and shaking at 180 r/m on a shaker for 72 hours.

4. A method of making the compounds according to claim 3, wherein the process of purification is characterized by collecting the culture medium into a centrifuge tube and centrifuging at 5000 r/m for 30 minutes, prior to transferring supernatant into new tubes and adjusting pH to 2.8 and staying at room temperature over night; centrifuging the supernatant at room temperature and 8000 r/m for 25 minutes to precipitate which is followed by two extractions over 48 hours with pure methanol and filtered through a 0.22 μm membrane filter; diluting the filtrate with deionized water to methanol (v:v) concentration of 30%, and adjusting pH to 7.0, and gravitationally passing the filtrate through an Amino (NH₂) solid phase extraction (Agilent Technologies, Amino (NH₂)-Box, 6 ml tubes, 500 mg) at room temperature; rinsing the subsequent stationary phase at room temperature by 10 ml of gradient eluent of a mixture, which is prepared first with deionized water in pure methanol (v/v=50/50), followed by pure methanol, and third by formic acid/methanol v/v=0.5/99.5, then by formic acid/methanol v/v=1/99, and finally by formic acid/methanol v/v=2/98; collecting the eluent of the final rinse with formic acid/methanol v/v=2/98, and prior to gravitationally passing through a LC-18 solid phase extraction (Agilent Technologies, Supelclean LC-18, 3 ml tubes, 500 mg) at room temperature and adjusting pH to 7.0, drying with pure nitrogen blow, and diluting with deionized water to methanol (v:v) concentration of 30%; rinsing the subsequent stationary phase at room temperature with 9 ml of gradient eluent of methanol-water solution, which is prepared first with deionized water in pure methanol (v/v=70/30), followed by deionized water/pure methanol (v/v=60/40), third with deionized water/pure methanol v/v=50/50), forth with deionized water/pure methanol v/v=40/60), fifth with deionized water/pure methanol v/v=30/70), finally with deionized water/pure methanol v/v=20/80); collecting the eluents from solutions of deionized water/pure methanol v/v=50/50 and v/v=40/60, and prior to gravitationally passing through a LC-18 solid phase extraction (Agilent Technologies, Supelclean LC-18, 3 ml tubes, 500 mg) again at room temperature and drying with pure nitrogen blow, and diluting with deionized water to methanol (v:v) concentration of 30%; rinsing the subsequent stationary phase at room temperature with 30 ml of gradient eluent of methanol-water solution, which is prepared first with deionized water in pure methanol (v/v=64/36), followed by deionized water/pure methanol (v/v=62/38), third with deionized water/pure methanol v/v=60/40), forth with deionized water/pure methanol v/v=58/42), fifth with deionized water/pure methanol v/v=56/44), fifth with deionized water/pure methanol v/v=54/46), fifth with deionized water/pure methanol v/v=52/48), fifth with deionized water/pure methanol v/v=50/50), finally with deionized water/pure methanol v/v=48/52); wherein the eluent from deionized water/pure methanol v/v=60/40 contains compound Locillomycin-A and the eluent from deionized water/pure methanol v/v=56/44 contains compound Locillomycin-B and the eluent from deionized water/pure methanol v/v=52/48 contains compound Locillomycin-C and the eluents are separately dried by pure nitrogen blow and further vacuum dried to obtain final white powder-type compounds.

5. A method for analyzing three new compounds of cyclic lipopeptide antibiotic Locillomycin-A, Locillomycin-B and Locillomycin-C thereof, according to structural formulae (I): 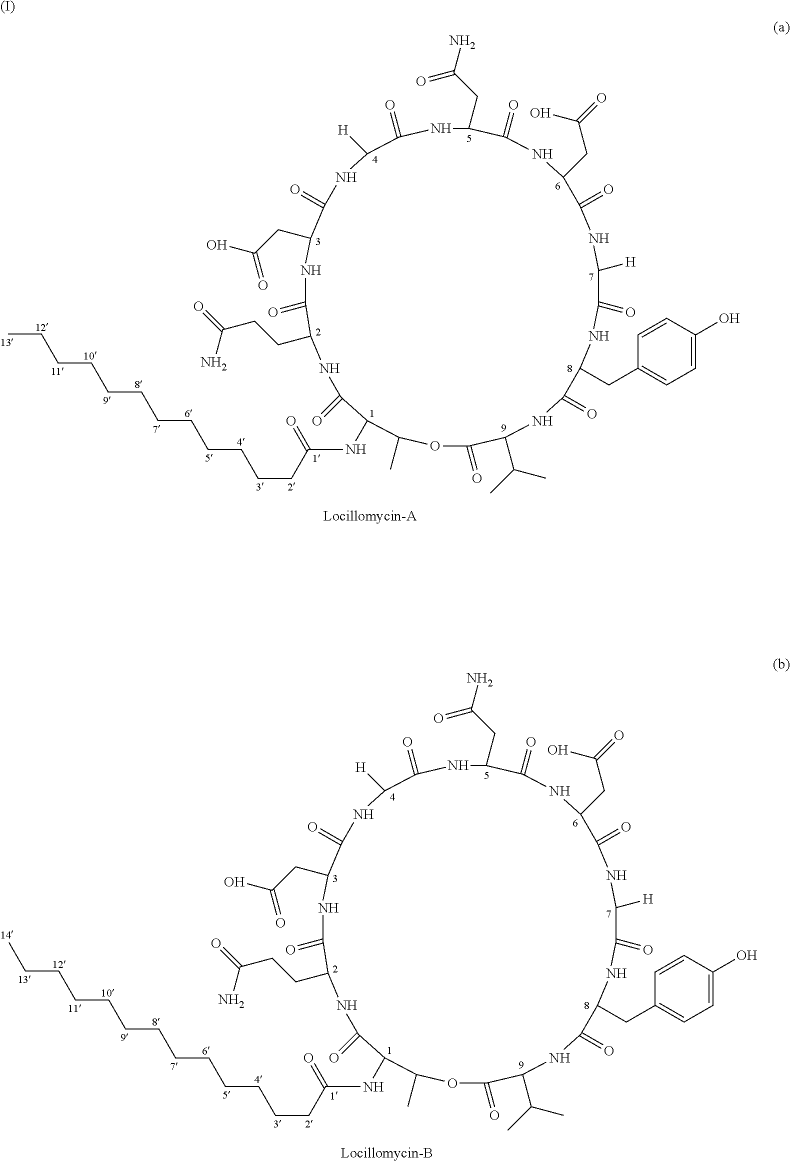 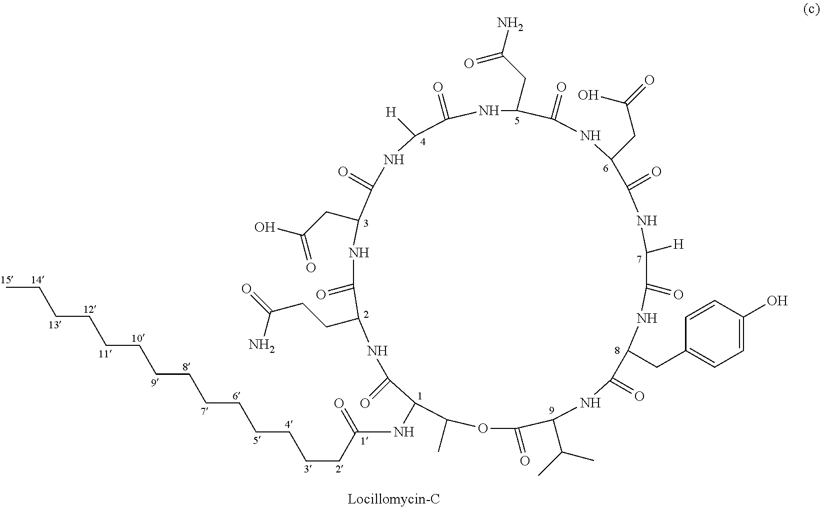
wherein:
1, or 2, or 3, or 4, or 5, or 6, or 7, or 8, or 9 is an amino acid residue thereof, 1: Thr; 2: Glu; 3, or 6: Asp; 4, or 7: Gly; 5: Asn; 8: Tyr; 9: Val; and
1′ to 13′, or 1′ to 14′, or 1′ to 15′ is a straight carbon chain wherein, the general chemical composition is CₙHₘNO, within which, the combinations of n and m can be one of the three: n=13 and m=25, or n=14 and m=27, or n=15 and m=29
characterized by using HPLC on a C-18 column (5 μm; 250 by 4.6 mm; VYDAC 218 TP; VYDAC, Hesperia, Calif.) with the acetonitrile-water-trifluoroacetic acid solvent system (50:50:0.5 [vol/vol/vol]) at a flow rate of 0.5 ml min⁻¹, wherein the retention times of 9.0 minutes, 13.0 minutes and 17.8 minutes and UV-visible spectrum at 230 nm are used to identify the Locillomycin-A, Locillomycin-B and Locillomycin-C respectively.

6. A method for analyzing three new compounds according to claim 5 characterized by a system analysis of data derived from UV spectra, amino acid identification, Edman degradation protein sequencing, ¹H-NMR, ¹³C-NMR, ¹³C-edited HSQC, HMBC, COSY, TOCSY, NOESY and/or ROESY.

7. A composition, comprising three new compounds of cyclic lipopeptide antibiotic Locillomycin-A, Locillomycin-B and Locillomycin-C thereof, according to structural formulae (I): 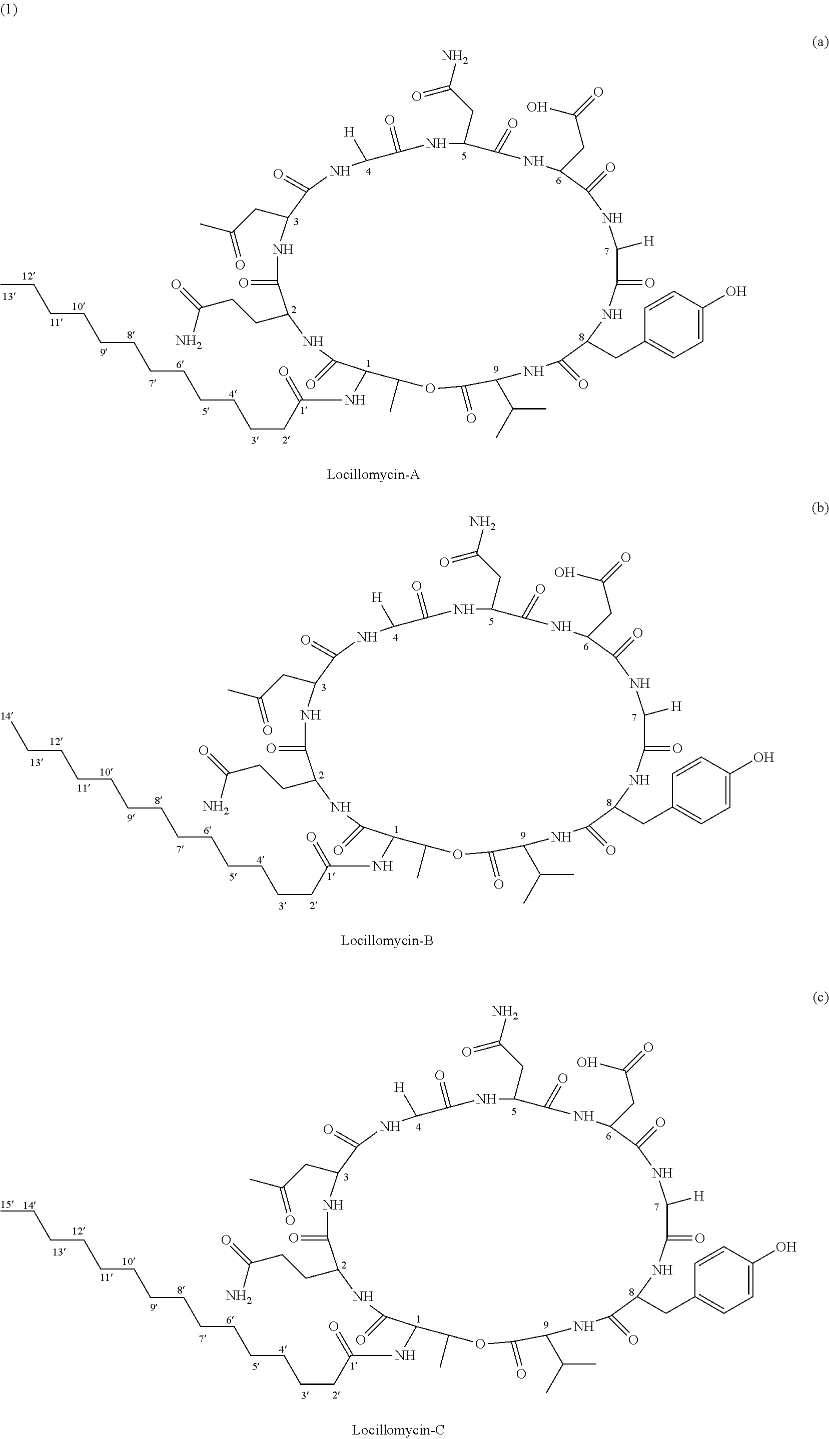 [SMILES: CCCCCCCCCCCCC(=O)NC1C(=O)NC(CCC(N)=O)C(=O)NC(CC(C)=O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C ; CCCCCCCCCCCCCC(=O)NC1C(=O)NC(CCC(N)=O)C(=O)NC(CC(C)=O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C ; CCCCCCCCCCCCCCC(=O)NC1C(=O)NC(CCC(N)=O)C(=O)NC(CC(C)=O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C]
wherein:
1, or 2, or 3, or 4, or 5, or 6, or 7, or 8, or 9 is an amino acid residue thereof, 1: Thr; 2: Glu; 3, or 6: Asp; 4, or 7: Gly; 5: Asn; 8: Tyr; 9: Val; and
1′ to 13′, or 1′ to 14′, or 1′ to 15′ is a straight carbon chain wherein, the general chemical composition is CₙHₘNO, within which, the combinations of n and m can be one of the three: n=13 and m=25, or n=14 and m=27, or n=15 and m=29.
and an acceptable carrier, excipient and diluent.

8. A method for treating a fungus, or a bacterium, or a virus, or combinations of fungus, bacterium and virus infections, comprising administering to a subject in need thereof three new compounds of cyclic lipopeptide antibiotic Locillomycin-A, Locillomycin-B and Locillomycin-C thereof, according to structural formulae (I): 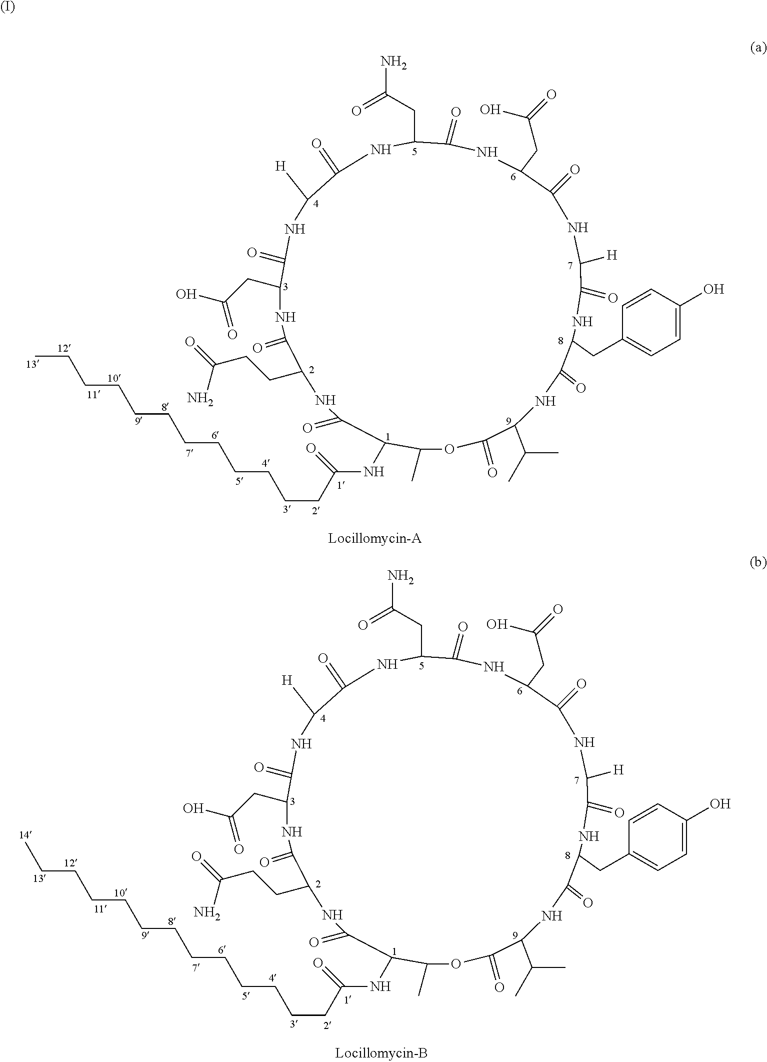 [SMILES: "(I)" ; CCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C ; CCCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C] 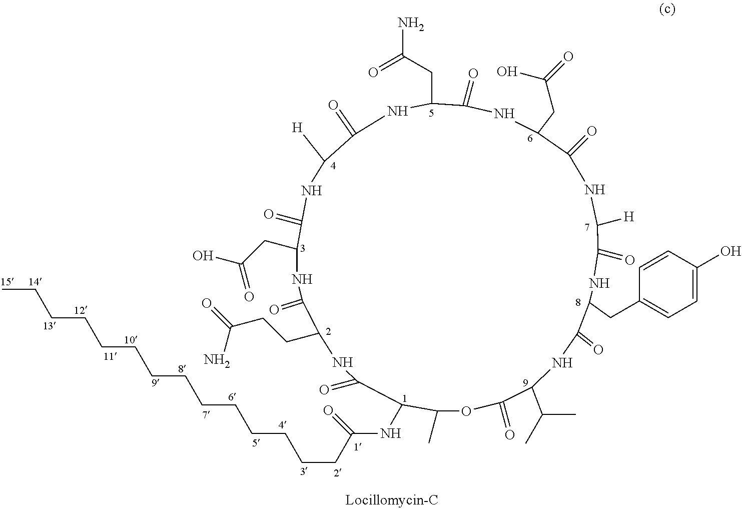 [SMILES: CCCCCCCCCCCCCCC(=O)NC1C(=O)NC(CCC(N)=O)C(=O)NC(CC(=O)O)C(=O)NCC(=O)NC(CC(N)=O)C(=O)NC(CC(=O)O)C(=O)NCC(=O)NC(Cc2ccc(O)cc2)C(=O)NC(C(C)C)C(=O)OC1C]
wherein:
1, or 2, or 3, or 4, or 5, or 6, or 7, or 8, or 9 is an amino acid residue thereof, 1: Thr; 2: Glu; 3, or 6: Asp; 4, or 7: Gly; 5: Asn; 8: Tyr; 9: Val; and
1′ to 13′, or 1′ to 14′, or 1′ to 15′ is a straight carbon chain wherein, the general chemical composition is CₙHₘNO, within which, the combinations of n and m can be one of the three: n=13 and m=25, or n=14 and m=27, or n=15 and m=29.


SEQUENCE LISTING (27 sequences)

SEQ ID NO: 1 - protein, 9 aa, Bacillus subtilis
TQDGNDGYV

SEQ ID NO: 2 - protein, 4 aa, Artificial Sequence
  Description of Artificial Sequence Synthetic peptide
DGND

SEQ ID NO: 3 - protein, 4 aa, Artificial Sequence
  Description of Artificial Sequence Synthetic peptide
QDGN

SEQ ID NO: 4 - protein, 5 aa, Artificial Sequence
  Description of Artificial Sequence Synthetic peptide
DGNDG

SEQ ID NO: 5 - protein, 5 aa, Artificial Sequence
  Description of Artificial Sequence Synthetic peptide
GNDGT

SEQ ID NO: 6 - protein, 5 aa, Artificial Sequence
  Description of Artificial Sequence Synthetic peptide
QDGND

SEQ ID NO: 7 - protein, 6 aa, Artificial Sequence
  Description of Artificial Sequence Synthetic peptide
QDGNDG

SEQ ID NO: 8 - protein, 6 aa, Artificial Sequence
  Description of Artificial Sequence Synthetic peptide
DGNDGY

SEQ ID NO: 9 - protein, 4 aa, Artificial Sequence
  Description of Artificial Sequence Synthetic peptide
VTQD

SEQ ID NO: 10 - protein, 4 aa, Artificial Sequence
  Description of Artificial Sequence Synthetic peptide
YVTQ

SEQ ID NO: 11 - protein, 7 aa, Artificial Sequence
  Description of Artificial Sequence Synthetic peptide
DGNDGYV

SEQ ID NO: 12 - protein, 5 aa, Artificial Sequence
  Description of Artificial Sequence Synthetic peptide
DGYVT

SEQ ID NO: 13 - protein, 7 aa, Artificial Sequence
  Description of Artificial Sequence Synthetic peptide
QDGNDGT

SEQ ID NO: 14 - protein, 8 aa, Artificial Sequence
  Description of Artificial Sequence Synthetic peptide
QDGNDGYV

SEQ ID NO: 15 - protein, 6 aa, Artificial Sequence
  Description of Artificial Sequence Synthetic peptide
YVTQDG

SEQ ID NO: 16 - protein, 7 aa, Artificial Sequence
  Description of Artificial Sequence Synthetic peptide
TQDGNDG

SEQ ID NO: 17 - protein, 7 aa, Artificial Sequence
  Description of Artificial Sequence Synthetic peptide
DGYVTQD

SEQ ID NO: 18 - protein, 7 aa, Artificial Sequence
  Description of Artificial Sequence Synthetic peptide
VTQDGND

SEQ ID NO: 19 - protein, 4 aa, Artificial Sequence
  Description of Artificial Sequence Synthetic peptide
DGYD

SEQ ID NO: 20 - protein, 5 aa, Artificial Sequence
  Description of Artificial Sequence Synthetic peptide
GNDGY

SEQ ID NO: 21 - protein, 4 aa, Artificial Sequence
  Description of Artificial Sequence Synthetic peptide
GYVT

SEQ ID NO: 22 - protein, 7 aa, Artificial Sequence
  Description of Artificial Sequence Synthetic peptide
QDGNDGY

SEQ ID NO: 23 - protein, 6 aa, Artificial Sequence
  Description of Artificial Sequence Synthetic peptide
NDGYVT

SEQ ID NO: 24 - protein, 7 aa, Artificial Sequence
  Description of Artificial Sequence Synthetic peptide
GNDGYVT

SEQ ID NO: 25 - protein, 4 aa, Artificial Sequence
  Description of Artificial Sequence Synthetic peptide
GNDG

SEQ ID NO: 26 - protein, 4 aa, Artificial Sequence
  Description of Artificial Sequence Synthetic peptide
NDGY

SEQ ID NO: 27 - protein, 5 aa, Artificial Sequence
  Description of Artificial Sequence Synthetic peptide
TQDGN